(19) United States
(12) Patent Application Publication
(10) Pub. No.: US 2024/0408177 A1
(43) Pub. Date: Dec. 12, 2024

(54) INTERLEUKIN-10 MUTEINS AND FUSION PROTEINS THEREOF

(71) Applicants: Hung-Kai CHEN, Los Altos, CA (US); Ellxiron Immunotherapeutics (Hong Kong) Limited, Central Hong Kong (CN)

(72) Inventors: Hung-Kai CHEN, Los Altos, CA (US); Po-Hao Chang, New Taipei City (TW); Wei Huang, New Taipei City (TW); Jing-Yi Huang, New Taipei City (TW); Pandelakis Andreas KONI, Larkspur, CA (US); Tsung-Hao CHANG, Tainan City (TW); Shih-Rang YANG, New Taipei City (TW); Yin-Ping WANG, New Taipei City (TW)

(21) Appl. No.: 18/698,803

(22) PCT Filed: Oct. 6, 2022
(86) PCT No.: PCT/US2022/077660

§ 371 Date: Apr. 5, 2024

Related U.S. Application Data
(60) Provisional application No. 63/252,772, filed on Oct. 6, 2021.

(51) Int. Cl. A61K 38/20; A61K 38/17; C07K 14/54; C07K 14/71

(52) U.S. Cl. CPC A61K 38/2066; A61K 38/179; C07K 14/5428; C07K 14/71; C07K 2319/30

(57) ABSTRACT

The present disclosure provides IL-10 muteins and use of IL-10 muteins in fusion proteins. The IL-10 mutein or the fusion protein comprise one or more substitution on amino acids in position 104, position 107, and a combination thereof, relative to amino acids of wild-type IL-10. Advantageously, the IL-10 mutein or the fusion protein thereof are provided with reduced aggregation potency during purification and extended half-life.

### CROSS REFERENCE TO RELATED APPLICATIONS

This application claims the benefit under 35 U.S.C. § 119 (e) of U.S. Provisional Patent Application Ser. No. 63/252,772, filed on Oct. 6, 2021, the entirety of which is incorporated herein by reference.

### FIELD OF THE INVENTION

The present disclosure relates biological active muteins of interleukin-10 (IL-10) and fusion proteins comprising the same with reduced aggregation and extended half-life.

### REFERENCE TO SEQUENCE LISTING

The official copy of the Sequence Listing is submitted concurrently with the specification as an ASCII formatted text file via EFS-Web, with a file name of “09793-006WO1_SeqList_ST25.txt”, a creation date of May 12, 2021, and a size of 234,722 bytes. The Sequence Listing filed via EFS-Web is part of the specification and is incorporated in its entirety by reference herein.

### BACKGROUND OF THE INVENTION

Interleukin-10 (IL-10) is a 35 kD homodimer that is composed of two non-covalently bonded monomers¹. Dimerization of IL-10 is strictly required for biological activity. Each monomer is composed of six helices with two disulfide bridges existing within the monomer (C30-C126 and C80-C132). The sequence of mature wild-type human IL-10 monomeric domain comprises an amino acid sequence having at least 80%, preferably at least 90%, and more preferably at least 95%, identity with SEQ ID NO: 2.

IL-10 exerts its innate and adaptive immune effects through binding to a cell surface IL-10 receptor (IL-10R) comprised of two IL-10Rα and two IL-10Rβ subunits. Binding of IL-10 to the IL-10R results in activation of JAK1 which in turn induces STAT3 phosphorylation.

IL-10 exerts potent anti-inflammatory effects by reducing antigen presentation through decreasing major histocompatibility complex expression and inhibiting production of proinflammatory cytokines from many cells including monocyte and macrophages. Hence, IL-10 has been a potential candidate to treat autoimmune disorders and has been investigated in clinical trials for ulcerative colitis and Crohn's disease. In addition, however, IL-10 also presents several immunostimulatory properties (e.g., stimulation of CD8 T cells) and there have been attempts to exploit this for cancer therapy¹⁻⁴. For example, a half-life extended form of IL-10 (pegylated IL-10) allowed for effective tumor control in mouse tumor models in a CD8⁺ T cell-dependent manner⁵, albeit pegylated IL-10 had to be dosed twice per day presumably because of its still short half-life. Also, pegylated IL-10 is well tolerated in cancer patient clinical trials, inducing CD8⁺ T cell immunity including elevation of CD8⁺ T cell proliferation, IFN-γ and granzyme B⁶. Having said this, pegylated IL-10 has not yet been proven to cause meaningful benefit in late-stage clinical trials above and beyond that of current standards of care.

More recent studies have revealed the ability of another half-life-extended form of IL-10, an immunoglobulin Fc domain fusion protein (IL10-Fc), to act directly on terminally-exhausted CD8⁺ T cells to restore metabolic capacity, causing re-acquisition of effector functions and proliferative expansion, and thereby boosting anti-tumor activity in mouse tumor models⁷. While this IL10-Fc demonstrated the utility of such a half-life-extended form of IL-107, it should be noted that this study used peri-tumoral administration of the IL10-Fc⁷, presumably because a challenge remains in optimizing the “therapeutic index” of IL-10 for optimal tumor control with minimal immuno-suppressive, adverse side effects.

There have been different attempts to optimize efficacy and reduce toxicity of IL-10 or extend half-life of IL-10 to avoid frequent dosing. For example, fusion of IL-10 to Fc to extend half-life in preclinical studies⁷. Fusion of anti-CD86 antibody to IL-10 to selectively triggering IL-10 receptor signaling on antigen presenting cells or fusion of antibody fragment F8 to IL-10 to target arthritic joints in RA patients⁸⁻⁹. Combining two or more different proteins into one fusion molecule can lead to manufacturing challenges such as aggregation during the cell culture or purification process. As a dimer with domain swapping property¹⁰, the helix structure of IL-10 dimer has placed a challenge in the pharmaceutical development of IL-10. High molecular weight molecules have been observed for IL-10 and IL-10 fusion proteins when expressed in eukaryotic system^{7,10}. The tendency to aggregate during production will be a challenge in the pharmaceutical development of IL-10.

A strategy to improve IL-10 therapeutic potential is to use IL-10 muteins with altered activity. Single substitution of amino acid isoleucine at position 87 to alanine or phenylalanine at position 111 to serine reduces IL10 activityl^{11,12}. IL-10 muteins with increased binding affinity of IL-10 to IL-10Rβ are found in yeast display screenings^{13,14}. Super 10, an IL-10 mutein with four substitutions, showed increased binding affinity to IL-10Rβ¹². It was proposed that substitution of amino acid asparagine at position 18 to tyrosine (N18Y) and arginine at position 104 to tryptophan (R104W) in Super 10 both enhanced the hydrophobic interaction to IL-10Rβ, and resulted in better STAT3 activation in some selected cell types¹³.

In summary, the foregoing studies all show substitutions for enhanced or decreased IL-10 signaling activity but not to improve manufacturability. IL-10 muteins with improved manufacturability, especially when the IL-10 mutein is fused to another molecule, would be an enormous practical value in the development of IL-10-based therapy.

### SUMMARY OF THE INVENTION

Investigations of IL-10 in animal models and in human patients both support a significant role of IL-10 in inflammatory, malignant and autoimmune diseases, and support the potential of IL-10 use in therapy if a sufficiently stable and amenable form can be developed.

The presented disclosure provides biological active IL-10 muteins with improved manufacturability, especially when IL-10 is fused to a polypeptide moiety. These IL-10 muteins with reduced IL-10 receptor binding but comparable biological activity can be used to design drugs with different pharmacokinetic characteristics or specific target cell selectivity.

In a first aspect, there is provided an IL-10 mutein with reduced aggregation potency during purification and extended half-life, comprising one or more substitution on amino acids in position 104, position 107, and a combination thereof, relative to amino acids of wild-type IL-10.

In one embodiment, the IL-10 mutein may comprise an amino acid sequence having at least 70%, 75%, 80%, 85%, 90%, 91%, 92%, 93%, 94%, 95%, 96%, 97%, 98%, or 99% sequence identity to SEQ ID NO: 2, and further include an amino acid substitution corresponding an amino acid residue selected from the group consisting of R104 and R107 of SEQ ID NO: 2.

In some embodiments, the one or more amino acid substitution is independently selected from the group consisting of an alanine substitution, an aspartic acid substitution, a glutamic acid substitution, a glutamine substitution, and combinations of any thereof.

In one embodiment, the substitution may comprise:
(1) R104Q;
(2) any one of R107A, R107E, R107Q and R107D; or
(3) a combination thereof.

In some embodiments, the IL-10 mutein of the disclosure include an amino acid sequence having at least 70%, 75%, 80%, 85%, 90%, 91%, 92%, 93%, 94%, 95%, 96%, 97%, 98%, or 99% sequence identity to SEQ ID NO: 2, and further include the amino acid substitutions corresponding to the following amino acid substitutions: (a) R104Q; (b) R107A; (c) R107E; (d) R107Q; (e) R107D; (f) R104Q/R107A; (g) R104Q/R107E; (h) R104Q/R107Q; and (i) R104Q/R107D.

In one embodiment, the IL-10 mutein may be monomer or dimer.

In one embodiment, The IL-10 mutein may further comprise a signal peptide. Preferably, the signal peptide may comprise amino acid sequence of SEQ ID NO: 14.

In at least one embodiment, the present disclosure provides a fusion protein, comprising a polypeptide which may bind to a target protein, wherein the polypeptide comprises an antibody or a fragment thereof, an antagonist, a receptor or a ligand of the target protein, or a protein-Trap; and the IL-10 mutein of the above fused to the polypeptide.

In one embodiment, the polypeptide may be fused to the IL-10 mutein via a linker.

In one embodiment, the linker may comprise the amino acid sequence of SEQ ID NO: 15-20.

In one embodiment, the IL-10 mutein may be fused to N-terminal or C-terminal of the polypeptide.

In one embodiment, the IL-10 mutein may be monomer or dimer.

In one embodiment, the fusion protein may comprise an amino acid sequence having at least 80% identity to:
(1) SEQ ID NO: 13;
(2) SEQ ID NO: 21 and SEQ ID NO: 22;
(3) SEQ ID NO: 23 and SEQ ID NO: 24;
(4) SEQ ID NO: 25 and SEQ ID NO: 26;
(5) SEQ ID NO: 27 and SEQ ID NO: 28; or
(6) SEQ ID NO: 29.

In one embodiment, the protein-Trap comprises vascular endothelial growth factor-Trap (VEGF-Trap) including aflibercept. In one preferred embodiment, the fusion protein may comprise an amino acid sequence having at least 80% identity to SEQ ID NO: 29.

In one embodiment, (i) the antibody is a human, humanized, or chimeric antibody; (ii) the antibody is a full length antibody of class IgG, optionally, wherein the class IgG antibody has an isotype selected from IgG1, IgG2, IgG3, and IgG4; (iii) the antibody comprises an Fc region variant, optionally an Fc region variant that alters effector function and/or a variant that alters antibody half-life; (iv) the antibody is an antibody fragment, optionally selected from the group consisting of F(ab′)₂, Fab′, Fab, Fv, single domain antibody (VHH), and scFv; (v) the antibody comprises an immunoconjugate, optionally, wherein the immunoconjugate comprises a therapeutic agent for treatment of a CSF1R-mediated, PDL1-mediated, PD1-mediated or VEGF-mediated disease or condition; or (vi) the antibody is a multi-specific antibody, optionally a bispecific antibody.

In one embodiment, the polypeptide may be a half-life extending moiety. In a preferred embodiment, the half-life extending moiety comprising IgG constant domain or fragment thereof, human serum albumin (HSA), or albumin-binding polypeptides or residue.

In one embodiment, the polypeptide may enable IL-10 dimer formation.

In a preferred embodiment of the present disclosure, the IL10 muteins fused to a molecule via N-terminus or C-terminus; preferably, this molecule is the fragment crystallizable region (Fc) of an antibody.

In preferred embodiments, the Fc portion is derived from a human immunoglobulin heavy chain, for example, IgG1, IgG2, IgG3, IgG4, or other classes; preferably, this Fc is derived from human IgG1 and IgG4; preferably, this Fc has mutations to modulate Fc effector function.

In a preferred embodiment of the present disclosure, the antibody or antigen-binding fragment thereof is selected from the group consisting of full length antibody, a chimeric antibody, Fab′, fab, F(ab′) 2, a bispecific antibody.

The present disclosure also provides embodiments of isolated polynucleotide or vector encoding the IL-10 mutein of the above or the fusion protein of the above.

In at least one embodiment the present disclosure provides an isolated host cell comprising the isolated polynucleotide or vector of the above; optionally, wherein the host cell is selected from a group consisting of Chinese hamster ovary (CHO) cell, a myeloma cell comprising Y0, NS0 or Sp2/0, a monkey kidney cell comprising COS-7, a human embryonic kidney line comprising 293, a baby hamster kidney cell (BHK), a mouse Sertoli cell comprising TM4, an African green monkey kidney cell comprising VERO-76, a human cervical carcinoma cell (HELA), a canine kidney cell, a human lung cell comprising W138, a human liver cell comprising Hep G2, a mouse mammary tumor cell, a TR1 cell, a Medical Research Council 5 (MRC 5) cell, and a FS4 cell.

In at least one embodiment, the present disclosure provides a method of producing an IL-10 mutein or a fusion protein, comprising culturing the host cell of the above so that an IL-10 mutein or a fusion protein is produced.

In at least one embodiment, the present disclosure provides a pharmaceutical composition comprising an IL-10 mutein of the above or a fusion protein of the above, and a pharmaceutically acceptable carrier, diluent or excipient.

In at least one embodiment, the present disclosure provides a use of an IL-10 mutein of the above or a fusion protein of the above for the manufacture of a medicament.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE FIGURES

FIG. 1 depicts sequences of precursor and mature form of human IL-10.

FIG. 2A depicts the sequence of IL-10 muteins with one substitution in each (highlighted). FIG. 2B depicts the sequence of IL-10 muteins with two substitutions in each (highlighted).

FIG. 3 depicts the sequence of wild-type IL10-Fc. IL-10 sequence is italicized, linker is underlined, human IgG1 Fc is in bold.

FIG. 4 depicts the results of non-reducing SDS-PAGE of wild-type IL10-Fc and various mutein IL10-Fc fusion proteins.

FIG. 5A depicts the chromatograms of size exclusion HPLC of IL10 (WT)-Fc and various mutein IL10-Fc fusion proteins (with one substitution) along with an analysis of percent peak area (% Area) of the high molecular (HMW), monomer and low molecular weight (LMW) peak. FIG. 5B depicts total versus monomer protein yield of IL10 (WT)-Fc and various mutein IL10-Fc fusion proteins produced using a CHO—S standardized transient protein expression method. FIG. 5C depicts the chromatogram of gel filtration for IL10 (WT)-Fc and IL10 (R104Q/R107A)-Fc by ÄKTA purifier 10 using Superdex 200 10/300 GL in the flow rate of 0.5 ml/min.

FIGS. 6A and 6B depict the result of recombinant human IL-10Rα binding of IL10 (WT)-Fc and various mutein IL10-Fc fusion proteins. FIG. 6C depicts the result of recombinant human IL-10Rβ binding of IL10 (WT)-Fc and various mutein IL10-Fc fusion proteins.

FIG. 7 depicts the influence of freeze-thawing (5 cycles) on binding affinity of IL10 (WT)-Fc and IL10 (R104Q)-Fc to human IL-10Rα.

FIG. 8 depicts the result of STAT3 activation induced by IL10 (WT)-Fc and various mutein IL10-Fc fusion proteins in HeLa IL10Rα-STAT3 luciferase reporter cells.

FIG. 9 depicts the result of granzyme B secretion induced by IL10 (WT)-Fc and various mutein IL10-Fc fusion proteins in human CD8+ T cells.

FIG. 10A depicts the sequence of IL10-Fc-VEGF TRAP. IL10: Lower Case, Linker: UPPERCASE ITALICS, Fc: UPPERCASE, VEGF Trap: UPPERCASE in Bold. FIG. 10B depicts the result of monomer yield of IL10 (WT)-Fc-VEGF TRAP and IL10 (R104Q/R107A)-Fc-VEGF TRAP fusion proteins.

FIG. 11 depicts the sequence of IL10 fusion proteins. (A) 6D4H22-IL10 fusion protein; (B) Bevacizumab-IL10; (C) YP7G-IL10; and (D) Avelumab-IL10; variable domain: UPPERCASE, constant domain: lower case, linker: UPPERCASE ITALICS, IL-10: UPPERCASE UNDERLINED.

FIG. 12 depicts the result of size exclusion chromatography or yield of various IL-10 fusion proteins. (A) the percent peak area (% Area) of HMW and monomer for various 6D4H22-IL10 fusion proteins relative to total protein content derived from results of size exclusion HPLC, chromatography examples of which are shown in (B).

FIG. 13 depicts the result of recombinant (A) human IL-10Rα binding and (B) mouse IL-10Rα binding of the 6D4H22-IL10 wilt-type and various 6D4H22-IL10 mutein fusion proteins.

FIG. 14 depicts the result of STAT3 activation induced by 6D4H22-IL10 (WT) versus various 6D4H22-IL10 mutein fusion proteins.

FIG. 15 depicts the result of granzyme B secretion induced by 6D4H22-IL10 (WT) versus various 6D4H22-IL10 mutein fusion proteins in human CD8+ T cells.

FIG. 16A depicts the size exclusion chromatography result of Bevacizumab-IL10 (WT) and various Bevacizumab-IL-10 mutein fusion proteins. FIGS. 16B and 16C depict the purification yield of monomers of various YP7G-IL10 and Avelumab-IL10 fusion proteins.

FIG. 17 depicts the concentration-time profiles of IL10 (WT)-Fc and various IL10-mutein-Fc fusion proteins in mice after intravenous administration.

### DETAILED DESCRIPTION

The present disclosure generally relates to novel IL-10 muteins and fusion proteins that provide reduced aggregation potency during purification and extended serum half-life. As described in greater details below, the IL-10 muteins comprises at least one amino acid substitution at positions 104, 107 or combination.

### Overview of Terminology and Techniques

Generally, the nomenclature used herein and the techniques and procedures described herein include those that are well understood and commonly employed by those of ordinary skill in the art, such as the common techniques and methodologies described in Sambrook et al., Molecular Cloning-A Laboratory Manual (2nd Ed.), Vols. 1-3, Cold Spring Harbor Laboratory, Cold Spring Harbor, N.Y., 1989 (hereinafter “Sambrook”); Current Protocols in Molecular Biology, F. M. Ausubel et al., eds., Current Protocols, a joint venture between Greene Publishing Associates, Inc. and John Wiley & Sons, Inc. (supplemented through 2011) (hereinafter “Ausubel”); Antibody Engineering, Vols. 1 and 2, R. Kontermann and S. Dubel, eds., Springer-Verlag, Berlin and Heidelberg (2010); Monoclonal Antibodies: Methods and Protocols, V. Ossipow and N. Fischer, eds., 2nd Ed., Humana Press (2014); Therapeutic Antibodies: From Bench to Clinic, Z. An, ed., J. Wiley & Sons, Hoboken, N.J. (2009); and Phage Display, Tim Clackson and Henry B. Lowman, eds., Oxford University Press, United Kingdom (2004).

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

Unless defined otherwise, all technical and scientific terms used herein have the same meaning as commonly understood by one of ordinary skill in the art to which the present invention pertains. It is to be understood that the terminology used herein is for the purpose of describing particular embodiments only and is not intended to be limiting. For purposes of interpreting this disclosure, the following description of terms will apply and, where appropriate, a term used in the singular form will also include the plural form and vice versa.

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding (i) either or (ii) both of those included limits are also included in the invention. For example, “1 to 50,” includes “2 to 25,” “5 to 20,” “25 to 50,” “1 to 10,” etc.

As used herein, the terms “variant” or “mutein” of an IL-10 polypeptide refers to a polypeptide in which one or more amino acid substitutions, deletions, and/or insertions are present as compared to the amino acid sequence of a reference IL-10 polypeptide, e.g., a wild-type IL-10 polypeptide. As such, the term “IL-10 polypeptide variant” includes naturally occurring allelic variants or alternative splice variants of an IL-10 polypeptide. For example, a polypeptide variant includes the substitution of one or more amino acids in the amino acid sequence of a parent IL-10 polypeptide with a similar or homologous amino acid(s) or a dissimilar amino acid(s).

The term “operably linked”, as used herein, denotes a physical or functional linkage between two or more elements, e.g., polypeptide sequences or polynucleotide sequences, which permits them to operate in their intended fashion. For example, an operably linkage between a polynucleotide of interest and a regulatory sequence (for example, a promoter) is functional link that allows for expression of the polynucleotide of interest. It should be understood that, operably linked” elements may be contiguous or non-contiguous. In the context of a polypeptide, “operably linked” refers to a physical linkage (e.g., directly or indirectly linked) between amino acid sequences (e.g., different domains) to provide for a described activity of the polypeptide. In the present disclosure, various domains of the recombinant polypeptides of the disclosure may be operably linked to retain proper folding, processing, targeting, expression, binding, and other functional properties of the recombinant polypeptides in the cell. Operably linked domains of the recombinant polypeptides of the disclosure may be contiguous or non-contiguous (e.g., linked to one another through a linker).

The term “polynucleotide” refers to a biopolymer composed of nucleotide monomers covalently bonded in a chain. The examples of polynucleotide comprise DNA (deoxyribonucleic acid) and RNA (ribonucleic acid). The polynucleotide may be delivered to the subject in need in a way known to the art so as to express the protein of interest, e.g. IL-10 muteins or fusion proteins thereof, in the subject in need directly.

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See, e.g., the NCBI web site at ncbi.nlm.nih.gov/BLAST. Such sequences are then said to be “substantially identical.” This definition also refers to, or may be applied to, the complement of a sequence. This definition also includes sequences that have deletions and/or additions, as well as those that have substitutions. Sequence identity can be calculated using published techniques and widely available computer programs, such as the GCS program package (Devereux et al, Nucleic Acids Res. 12:387, 1984), BLASTP, BLASTN, FASTA (Atschul et al, J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

The term “pharmaceutically acceptable carrier, diluent or excipient” as used herein refers to any suitable substance that provides a pharmaceutically acceptable carrier, additive or diluent for administration of a compound(s) of interest to a subject. As such, “pharmaceutically acceptable carrier, diluent or excipient” can encompass substances referred to as pharmaceutically acceptable diluents, pharmaceutically acceptable additives, and pharmaceutically acceptable carriers. As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics and additional therapeutic agents) can also be incorporated into the compositions.

The term “recombinant” or “engineered” nucleic acid molecule or polypeptide as used herein, refers to a nucleic acid molecule or polypeptide that has been altered through human intervention. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. As non-limiting examples, a recombinant nucleic acid molecule can be one which: 1) has been synthesized or modified in vitro, for example, using chemical or enzymatic techniques; 2) includes conjoined nucleotide sequences that are not conjoined in nature; 3) has been engineered using molecular cloning techniques such that it lacks one or more nucleotides with respect to the naturally occurring nucleic acid molecule sequence; and/or 4) has been manipulated using molecular cloning techniques such that it has one or more sequence changes or rearrangements with respect to the naturally occurring nucleic acid sequence. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. Another non-limiting example of a recombinant nucleic acid and recombinant protein is an IL-10 polypeptide variant as disclosed herein.

“IL10” or “IL-10,” as used herein, refers to the cytokine, interleukin 10, also known as cytokine synthesis inhibitory factor (CSIF), and is intended to also include naturally-occurring variants, engineered variants, and/or synthetically modified versions of interleukin 10 that retain its cytokine functions. Amino acid sequences of various exemplary IL10 polypeptides and recombinant IL10 fusion constructs are provided in Table 2 below and the attached Sequence Listing. Other exemplary engineered and/or modified IL10 polypeptides that retain cytokine functions are known in the art (see e.g., U.S. Pat. No. 7,749,490 B2; US 2017/0015747 A1; Naing, A. et al. “PEGylated IL-10 (Pegilodecakin) Induces Systemic Immune Activation, CD8+ T Cell Invigoration and Polyclonal T Cell Expansion in Cancer Patients.” Cancer Cell 34, 775-791.e3 (2018); Gorby, C. et al. “Engineered IL-10 variants elicit potent immunomodulatory effects at low ligand doses.” Sci Signal 13, (2020); Yoon, S. I. et al. “Epstein-Barr virus IL-10 engages IL-10R1 by a two-step mechanism leading to altered signaling properties.” J Biol Chem 287, 26586-26595 (2012)).

“Fusion protein,” as used herein, refers to two or more protein and/or polypeptide molecules that are linked (or “fused”) in a configuration that does not occur naturally. An exemplary fusion protein of the present disclosure includes the “IL10-Fc” fusion protein that comprises an IL10 polypeptide covalently linked through a polypeptide linker sequence at its C-terminus to an immunoglobulin Fc region polypeptide. Fusion proteins of the present disclosure also include “antibody fusions” that comprise a full-length IgG antibody (with both a heavy chain and a light chain polypeptide) that is covalently linked through a polypeptide linker sequence at its heavy chain C-terminus to an IL10 polypeptide.

“Polypeptide linker” or “linker sequence” as used herein refers to a chain of two or more amino acids with each end of the chain covalently attached to a different polypeptide molecule, thereby functioning to conjugate or fuse the different polypeptides. Typically, polypeptide linkers comprise polypeptide chains of 1 to 42 amino acids, preferably 5 to 30 amino acids. A wide range of polypeptide linkers are known in the art and can be used in the compositions and methods of the present disclosure. Exemplary polypeptide linkers include those shown in Table 1, and other specific linker sequences as disclosed elsewhere herein.

Table 1
Exemplary polypeptide linkers

| | | |
|---|---|---|
| Linker 1 | (GGGGS)n; n = 1-6 repeat(s) | SEQ ID NO: 15 |
| Linker 2 | (SSSSG)n; n = 1-6 repeat(s) | SEQ ID NO: 16 |
| Linker 3 | (G)n; n = 1-6 repeat(s) | SEQ ID NO: 17 |
| Linker 4 | (EAAAK)n; n = 1-6 repeat(s) | SEQ ID NO: 18 |
| Linker 5 | (XP)n; n = 1-6 repeat(s) | SEQ ID NO: 19 |
| Linker 6 | ENLYFQ(-G/S); n = 1-6 | SEQ ID NO: 20 |
| | repeat(s) | |

“Antibody,” as used herein, refers to a molecule comprising one or more polypeptide chains that specifically binds to, or is immunologically reactive with, a particular antigen. Exemplary antibodies of the present disclosure include monoclonal antibodies, polyclonal antibodies, chimeric antibodies, humanized antibodies, human antibodies, antibody fusions, multispecific antibodies (e.g., bispecific antibodies), monovalent antibodies (e.g., single-arm antibodies), multivalent antibodies, antigen-binding fragments (e.g., Fab′, F(ab′)₂, Fab, Fv, rlgG, and scFv fragments), and synthetic antibodies (or antibody mimetics).

“Full-length antibody,” “intact antibody,” or “whole antibody” are used herein interchangeably to refer to an antibody having a structure substantially similar to a native antibody structure or having heavy chains that contain an Fc region as defined herein.

“Antibody fragment” refers to a portion of a full-length antibody which is capable of binding the same antigen as the full-length antibody. Examples of antibody fragments include, but are not limited to, Fv, Fab, Fab′, Fab′-SH, F(ab′)₂; diabodies; linear antibodies; monovalent, or single-armed antibodies; single-chain antibody molecules (e.g., scFv); and multispecific antibodies formed from antibody fragments.

“Fc region,” refers to a dimer complex comprising the C-terminal polypeptide sequences of an immunoglobulin heavy chain, wherein a C-terminal polypeptide sequence is that which is obtainable by papain digestion of an intact antibody. The Fc region may comprise native or variant Fc sequences. Although the boundaries of the Fc sequence of an immunoglobulin heavy chain may vary, the human IgG heavy chain Fc sequence is usually defined to stretch from an amino acid residue at about position Cys226, or from about position Pro230, to the carboxyl-terminus of the Fc sequence. However, the C-terminal lysine (Lys447) of the Fc sequence may or may not be present. The Fc sequence of an immunoglobulin generally comprises two constant domains, a CH2 domain and a CH3 domain, and optionally comprises a CH4 domain.

“Antibody fusion” refers to an antibody that is covalently conjugated (or fused) to a polypeptide or protein, typically via a linker to a terminus of the antibody's light chain (LC) or heavy chain (HC). Exemplary antibody fusions of the present disclosure include an anti-CSFIR antibody fused to a recombinant IL10 polypeptide via a 14 amino acid polypeptide linker (e.g., SEQ ID NO: 21-22) from the C-terminus of the antibody heavy chain to the N-terminus of the IL10 polypeptide. Antibody fusions are labeled herein with an “antibody-polypeptide” nomenclature to indicate the fusion components, such as “Ab-IL10” or “anti CSFIR-IL10.” As described elsewhere herein, an antibody fusion of the present disclosure can include a full-length IgG antibody, comprising a dimeric complex of heavy chain-light chain pairs, where each heavy chain C-terminus is linked through a polypeptide linker sequence to an IL10 polypeptide.

The half-life extending moiety of the present disclosure can be covalently fused, attached, linked or conjugated to an IL-10 mutein. A half-life extending moiety can be, for example, a polymer, such as polyethylene glycol (PEG), a cholesterol group, a carbohydrate or oligosaccharide; a fatty acid or any natural or synthetic protein, polypeptide or peptide that binds to a salvage receptor. Preferably, the half-life extending moiety is covalently linked, optionally via a linker, to plasma protein (albumin and immunoglobulin) with long serum half-lives. In other embodiment, the half-life extending moiety is an albumin binding residue. An “Albumin binding residue” as used herein means a residue which binds non-covalently to human serum albumin. In one embodiment the albumin binding residue is a lipophilic residue. In another embodiment, the albumin binding residue is negatively charged at physiological pH. An albumin binding residue typically comprises a carboxylic acid which can be negatively charged. Examples of albumin binding residue includes fatty acids. In other embodiment, the half-life extending moiety is an IgG constant domain or fragment thereof (e.g., the Fc region), human serum albumin (HSA), or an albumin-binding polypeptides or residue such as for example a fatty acid. Preferably, the half-life extending moiety portion of the bioconjugate is a human serum albumin or an Fc region. Most preferably, the half-life extending moiety portion of the bioconjugate is an Fc region.

The half-life extending moiety is attached in such a way so as to enhance, and/or not to interfere with, the biological function of the constituent portions of the bio-conjugates of the present disclosure, e.g., IL-10 mutein of the present disclosure. In some embodiments, the IL-10 mutein of the present disclosure can be fused to a half-life extending moiety, optionally via a linker. The half-life extending moiety can be a protein such as an IgG constant domain or fragment thereof (e.g., the Fc region), human serum albumin (HSA), or albumin-binding polypeptides or residue (e.g. a fatty acid). Such proteins disclosed herein can also form multimers.

In some embodiments, the half-life extending moiety (e.g., HSA, Fc, fatty acid etc.) is covalently linked or fused to the N-terminus of the IL-10 mutein of the present disclosure. In other embodiments, the half-life extending moiety (e.g., HSA, Fc, fatty acid etc.) is covalently linked or fused to C-terminus of the IL-10 mutein of the present disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Detailed Description of Various Embodiments

### I. IL10 Muteins and Fusion Proteins Thereof

The human IL10 cytokine is a homodimeric protein of two polypeptide subunits. IL10 signals through an IL-10R consisting of two IL10 receptor-1 (IL-10Rα subunit) and two IL10 receptor-2 (IL-10Rβ subunit) proteins. Consequently, the functional receptor consists of four IL10 receptor molecules. Binding of IL10 to IL-10R induces STAT3 signaling via the phosphorylation of the cytoplasmic tails of IL10 receptor by JAK1 and Tyk2. IL-10 is primarily produced by monocytes and, to a lesser extent, lymphocytes, namely type-II T helper cells (T_{H}2), mast cells, CD4⁺CD25⁺Foxp3⁺ regulatory T cells, and in a certain subset of activated T cells and B cells. Table 2 below provides a summary description of the amino sequences of the human IL10 polypeptide and a recombinant IL10-Fc fusion construct used in the Examples of the present disclosure, and their sequence identifiers. The sequences also are included in the accompanying Sequence Listing

TABLE 2
Recombinant IL10 polypeptides and polypeptide linkers

| | | SEQ |
|---|---|---|
| Description | Sequence | ID NO: |
| IL-10 | MHSSALLCCLVLLTGVRASPGQGTQSENSCTHFPGNLPNML | 1 |
| precursor | RDLRDAFSRVKTFFQMKDQLDNLLLKESLLEDFKGYLGCQ | |
| | ALSEMIQFYLEEVMPQAENQDPDIKAHVNSLGENLKTLRLR | |
| | LRRCHRFLPCENKSKAVEQVKNAFNKLQEKGIYKAMSEFDI | |
| | FINYIEAYMTMKIRN | |
| IL-10 | SPGQGTQSENSCTHFPGNLPNMLRDLRDAFSRVKTFFQMKD | 2 |
| mature form | QLDNLLLKESLLEDFKGYLGCQALSEMIQFYLEEVMPQAEN | |
| | QDPDIKAHVNSLGENLKTLRLRLRRCHRFLPCENKSKAVEQ | |
| | VKNAFNKLQEKGIYKAMSEFDIFINYIEAYMTMKIRN | |
| IL-10 | SPGQGTQSENSCTHFPGNLPNMLRDLRDAFSRVKTFFQMKD | 3 |
| R104Q | QLDNLLLKESLLEDFKGYLGCQALSEMIQFYLEEVMPQAEN | |
| | QDPDIKAHVNSLGENLKTLRLQLRRCHRFLPCENKSKAVEQ | |
| | VKNAFNKLQEKGIYKAMSEFDIFINYIEAYMTMKIRN | |
| IL-10 | SPGQGTQSENSCTHFPGNLPNMLRDLRDAFSRVKTFFQMKD | 4 |
| R107A | QLDNLLLKESLLEDFKGYLGCQALSEMIQFYLEEVMPQAEN | |
| | QDPDIKAHVNSLGENLKTLRLRLRACHRFLPCENKSKAVEQ | |
| | VKNAFNKLQEKGIYKAMSEFDIFINYIEAYMTMKIRN | |
| IL-10 | SPGQGTQSENSCTHFPGNLPNMLRDLRDAFSRVKTFFQMKD | 5 |
| R107E | QLDNLLLKESLLEDFKGYLGCQALSEMIQFYLEEVMPQAEN | |
| | QDPDIKAHVNSLGENLKTLRLRLRECHRFLPCENKSKAVEQ | |
| | VKNAFNKLQEKGIYKAMSEFDIFINYIEAYMTMKIRN | |
| IL-10 | SPGQGTQSENSCTHFPGNLPNMLRDLRDAFSRVKTFFQMKD | 6 |
| R107Q | QLDNLLLKESLLEDFKGYLGCQALSEMIQFYLEEVMPQAEN | |
| | QDPDIKAHVNSLGENLKTLRLRLRQCHRFLPCENKSKAVEQ | |
| | VKNAFNKLQEKGIYKAMSEFDIFINYIEAYMTMKIRN | |
| IL-10 | SPGQGTQSENSCTHFPGNLPNMLRDLRDAFSRVKTFFQMKD | 7 |
| R107D | QLDNLLLKESLLEDFKGYLGCQALSEMIQFYLEEVMPQAEN | |
| | QDPDIKAHVNSLGENLKTLRLRLRDCHRFLPCENKSKAVEQ | |
| | VKNAFNKLQEKGIYKAMSEFDIFINYIEAYMTMKIRN | |
| IL-10 | SPGQGTQSENSCTHFPGNLPNMLRDLRDAFSRVKTFFQMKD | 8 |
| K119S | QLDNLLLKESLLEDFKGYLGCQALSEMIQFYLEEVMPQAEN | |
| | QDPDIKAHVNSLGENLKTLRLRLRRCHRFLPCENKSSAVEQ | |
| | VKNAFNKLQEKGIYKAMSEFDIFINYIEAYMTMKIRN | |
| L-10 | SPGQGTQSENSCTHFPGNLPNMLRDLRDAFSRVKTFFQMKD | 9 |
| R104Q/R107A | QLDNLLLKESLLEDFKGYLGCQALSEMIQFYLEEVMPQAEN | |
| | QDPDIKAHVNSLGENLKTLRLQLRACHRFLPCENKSKAVEQ | |
| | VKNAFNKLQEKGIYKAMSEFDIFINYIEAYMTMKIRN | |
| L-10 | SPGQGTQSENSCTHFPGNLPNMLRDLRDAFSRVKTFFQMKD | 10 |
| R104Q/R107E | QLDNLLLKESLLEDFKGYLGCQALSEMIQFYLEEVMPQAEN | |
| | QDPDIKAHVNSLGENLKTLRLQLRECHRFLPCENKSKAVEQ | |
| | VKNAFNKLQEKGIYKAMSEFDIFINYIEAYMTMKIRN | |
| L-10 | SPGQGTQSENSCTHFPGNLPNMLRDLRDAFSRVKTFFQMKD | 11 |
| R104Q/R107Q | QLDNLLLKESLLEDFKGYLGCQALSEMIQFYLEEVMPQAEN | |
| | QDPDIKAHVNSLGENLKTLRLQLRQCHRFLPCENKSKAVEQ | |
| | VKNAFNKLQEKGIYKAMSEFDIFINYIEAYMTMKIRN | |
| L-10 | SPGQGTQSENSCTHFPGNLPNMLRDLRDAFSRVKTFFQMKD | 12 |
| R104Q/R107D | QLDNLLLKESLLEDFKGYLGCQALSEMIQFYLEEVMPQAEN | |
| | QDPDIKAHVNSLGENLKTLRLQLRDCHRFLPCENKSKAVEQ | |
| | VKNAFNKLQEKGIYKAMSEFDIFINYIEAYMTMKIRN | |
| Wild-type | SPGQGTQSENSCTHFPGNLPNMLRDLRDAFSRVKTFFQMKDQ | 13 |
| IL10-Fc | LDNLLLKESLLEDFKGYLGCQALSEMIQFYLEEVMPQAENQDP | |
| | DIKAHVNSLGENLKTLRLRLRRCHRFLPCENKSKAVEQVKNAFN | |
| | KLQEKGIYKAMSEFDIFINYIEAYMTMKIRNGGGGSGGGGSGG | |
| | GGSPKSCDKTHTCPPCPAPELLGGPSVFLFPPKPKDTLMI | |
| | SRTPEVTCVVVDVSHEDPEVKFNWYVDGVEVHNAKTKP | |
| | REEQYNSTYRVVSVLTVLHQDWLNGKEYKCKVSNKALP | |
| | APIEKTISKAKGQPREPQVYTLPPSRDELTKNQVSLTCLV | |
| | KGFYPSDIAVEWESNGQPENNYKTTPPVLDSDGSFFLYSK | |
| | LTVDKSRWQQGNVFSCSVMHEALHNHYTQKSLSLSPGK | |

IL-10: italicized
Linker: underlined
human IgG1 Fc fragment: Bold lower case

The term IL-10 or IL-10 polypeptide refers to wild-type IL-10, whether native or recombinant, and encompasses homologs, orthologs, variants, and fragments thereof, as well as IL-10 polypeptides having, for example, a leader sequence (e.g., a signal peptide). As such, an IL-10 polypeptide includes, but not limited to, a recombinantly produced IL-10 polypeptide, synthetically produced IL-10 polypeptide, and IL-10 polypeptide extracted from cells or tissues. As a non-limiting example of IL-10 polypeptides of the disclosure, an amino acid sequence of mature human IL-10 is depicted in SEQ ID NO: 2. Exemplary IL-10 homologs and modified forms thereof from other mammalian species include IL-10 polypeptides from rat (accession NP_036986.2; GI 148747382); cow (accession NP_776513.1; GI 41386772); sheep (accession NP_00 1009327.1; GI 57164347); dog (accession ABY86619.1; GI 166244598); and rabbit (accession AAC23839.1; GI 3242896). Further examples of IL-10 polypeptides suitable for introduction of amino acid substitutions described herein include, but are not limited to, virus-encoded IL-10 homologs, including IL-10 polypeptides from genera Cytomegalovirus, Lymphocryptovirus, Macavirus, Percavirus, Parapoxvirus, Capripoxvirus, and Avipoxvirus. Non-limiting examples of cytomegalovirus IL-10 polypeptides include those from human cytomegalovirus (accession AAR31656 and ACR49217), Green monkey cytomegalovirus (accession AEV80459), rhesus cytomegalovirus (accession AAF59907), baboon cytomegalovirus (accession AAF63436), owl monkey cytomegalovirus (accession AEV80800), and squirrel monkey cytomegalovirus (accession AEV80955). Examples of cytomegalovirus IL-10 polypeptides include those from Epstein-Barr virus (accession CAD53385), Bonobo herpesvirus (accession XP_003804206.1), Rhesus lymphocryptovirus (accession AAK95412), and baboon lymphocryptovirus (accession AAF23949). Additional information regarding viral IL-10 polypeptides and their control of host immune function can be found in, for example, Slobedman B. et al., J. Virol. October 2009, p. 9618-9629; and Ouyang P. et al., J. Gen. Virol. (2014), 95, 245-262.

An amino acid sequence of wild-type human IL-10 precursor polypeptide (e.g., pre-protein with a signal peptide) is depicted in SEQ ID NO: 1, which is a 178 amino acid residue protein with an N-terminal 18 amino acid signal peptide, depicted in SEQ ID NO: 14, that can be removed to generate a 160 amino acid mature protein of SEQ ID NO: 2. However, mature proteins are often used to generate recombinant polypeptide constructs. N-terminal variant of IL-10 has been reported (U.S. Pat. No. 5,328,989). Therefore, for the purpose of the present disclosure, all amino acid numbering is based on the mature polypeptide sequence of the IL-10 protein set forth in SEQ ID NO: 2.

In addition to the naturally-occurring human IL-10, a variety of engineered and/or synthetically modified IL-10 polypeptides that retain the cytokine functions of IL-10 are known in the art. The PEGylated IL-10, Pegilodecakin, has been shown to retain the anti-tumor immune surveillance function of naturally-occurring human IL-10. See, Naing, A. et al. “PEGylated IL-10 (Pegilodecakin) Induces Systemic Immune Activation, CD8+ T Cell Invigoration and Polyclonal T Cell Expansion in Cancer Patients.” Cancer Cell 34, 775-791. (2018). The engineered IL-10 variant R5A11 has been shown to have higher affinity to IL-10Rβ, exhibit enhanced signaling activities in human CD8+ T-cells, and enhances the anti-tumor function of CAR-T cells. See, Gorby, C. et al. “Engineered IL-10 variants elicit potent immunomodulatory effects at low ligand doses.” Sci Signal 13, (2020). The IL-10 from Epstein-Barr virus has weaker binding to the IL-10R1, but retains the immunosuppressive cytokine activities of human IL10, while having lost the ability to induce immunostimulatory activities with some cells. See, Yoon, S. I. et al. “Epstein-Barr virus IL-10 engages IL-10R1 by a two-step mechanism leading to altered signaling properties.” J Biol Chem 287, 26586-26595 (2012). U.S. Pat. No. 7,749,490B2 and US2017/0015747A1 described engineered IL-10 mutants (e.g., F129S-IL-10) that exhibit less immunostimulatory activity in MC/9 cell proliferation assay. Generally, it is contemplated that any engineered or modified version of IL-10 polypeptide that retains some IL-10 cytokine function can be used in any of the IL-10 muteins and fusion protein of the present disclosure.

Without being bound to any particular theory, the experimental data described herein supports that the manufacture, such as yields, of the IL-10 muteins are improved, especially when IL-10 is fused to another molecule. Also, the present disclosure also provides that the IL-10 muteins have better pharmacological properties over wild-type IL-10. Furthermore, IL-10 muteins with reduced IL-10 receptor binding but comparable biological activity are also provided and can be used to design drugs with different pharmacokinetic characteristics or target-cell selectivity.

### II. Pharmaceutical Compositions and Formulations of IL10 Mutein or Fusion Protein Thereof

The present disclosure also provides pharmaceutical compositions and pharmaceutical formulations comprising an IL10 mutein or fusion protein thereof. In some embodiments, the present disclosure provides a pharmaceutical formulation comprising an IL10 mutein or fusion protein thereof as described herein and a pharmaceutically acceptable carrier. Such pharmaceutical formulations can be prepared by mixing an IL10 mutein or fusion protein thereof, having the desired degree of purity, with one or more pharmaceutically acceptable carriers. Typically, such antibody formulations can be prepared as an aqueous solution (see e.g., U.S. Pat. No. 6,171,586, and WO2006/044908) or as a lyophilized formulation (see e.g., U.S. Pat. No. 6,267,958).

Pharmaceutically acceptable carriers are generally nontoxic to recipients at the dosages and concentrations employed. A wide range of such pharmaceutically acceptable carriers are well-known in the art (see e.g., Remington's Pharmaceutical Sciences 16th edition, Osol, A. Ed. (1980)). Exemplary pharmaceutically acceptable carriers useful in the formulations of the present disclosure can include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Pharmaceutically acceptable carriers useful in the formulations of the present disclosure can also include interstitial drug dispersion agents, such as soluble neutral-active hyaluronidase glycoproteins (sHASEGP) (see e.g., US Pat. Publ. Nos. 2005/0260186 and 2006/0104968), such as human soluble PH-20 hyaluronidase glycoproteins (e.g., rHuPH20 or HYLENEX®, Baxter International, Inc.).

It is also contemplated that the formulations disclosed herein may contain active ingredients in addition to the IL10 mutein or fusion protein thereof, as necessary for the particular indication being treated in the subject to whom the formulation is administered. Preferably, any additional active ingredient has activity complementary to that of the IL10 mutein or fusion protein thereof activity and the activities do not adversely affect each other.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

In some embodiments, the formulation can be a sustained-release preparation of the antibody and/or other active ingredients. Suitable examples of sustained-release preparations include semipermeable matrices of solid hydrophobic polymers containing the antibody, which matrices are in the form of shaped articles, e.g. films, or microcapsules.

Typically, the formulations of the present disclosure to be administered to a subject are sterile. Sterile formulations may be readily prepared using well-known techniques, e.g., by filtration through sterile filtration membranes.

In some embodiments of the methods of treatment of the present disclosure, the IL10 mutein or fusion protein thereof or pharmaceutical formulation comprising an IL10 mutein or fusion protein thereof is administered to a subject by any mode of administration that delivers the agent systemically, or to a desired target tissue. Systemic administration generally refers to any mode of administration of the antibody into a subject at a site other than directly into the desired target site, tissue, or organ, such that the antibody or formulation thereof enters the subject's circulatory system and, thus, is subject to metabolism and other like processes.

Accordingly, modes of administration useful in the methods of treatment of the present disclosure can include, but are not limited to, injection, infusion, instillation, and inhalation. Administration by injection can include intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebro spinal, and intrasternal injection and infusion.

In some embodiments, a pharmaceutical formulation of the IL10 mutein or fusion protein thereof is formulated such that the antibody is protected from inactivation in the gut. Accordingly, the method of treatments can comprise oral administration of the formulation.

In some embodiments, use of the compositions or formulations comprising an IL10 mutein or fusion protein thereof of the present disclosure as a medicament are also provided. Additionally, in some embodiments, the present disclosure also provides for the use of a composition or a formulation comprising an IL10 mutein or fusion protein thereof in the manufacture or preparation of a medicament, particularly a medicament for treating, preventing or inhibiting disease. In a further embodiment, the medicament is for use in a method for treating, preventing or inhibiting a disease comprising administering to an individual having a disease an effective amount of the medicament. In certain embodiments, the medicament further comprises an effective amount of at least one additional therapeutic agent, or treatment.

The appropriate dosage of the IL10 mutein or fusion protein thereof contained in the compositions and formulations of the present disclosure (when used alone or in combination with one or more other additional therapeutic agents) will depend on the specific disease or condition being treated, the severity and course of the disease, whether the antibody is administered for preventive or therapeutic purposes, the previous therapy administered to the patient, the patient's clinical history and response to the antibody, and the discretion of the attending physician. The IL10 mutein or fusion protein thereof included in the compositions and formulations described herein, can be suitably administered to the patient at one time, or over a series of treatments. Various dosing schedules including but not limited to single or multiple administrations over various time-points, bolus administration, and pulse infusion are contemplated herein.

Depending on the type and severity of the disease, about 1 μg/kg to 15 mg/kg of IL10 mutein or fusion protein thereof in a formulation of the present disclosure is an initial candidate dosage for administration to a human subject, whether, for example, by one or more separate administrations, or by continuous infusion. Generally, the administered dosage of the IL10 mutein or fusion protein would be in the range from about 0.001 mg/kg to about 10 mg/kg. In some embodiments, one or more doses of about 0.5 mg/kg, 2.0 mg/kg, 4.0 mg/kg or 10 mg/kg (or any combination thereof) may be administered to a patient.

Dosage administration can be maintained over several days or longer, depending on the condition of the subject, for example, administration can continue until the disease is sufficiently treated, as determined by methods known in the art. In some embodiments, an initial higher loading dose may be administered, followed by one or more lower doses. However, other dosage regimens may be useful. The progress of the therapeutic effect of dosage administration can be monitored by conventional techniques and assays.

Accordingly, in some embodiments of the methods of the present disclosure, the administration of the IL10 mutein or fusion protein thereof comprises a daily dosage from about 1 mg/kg to about 100 mg/kg. In some embodiments, the dosage of IL10 mutein or fusion protein thereof comprises a daily dosage of at least about 0.01 mg/kg, at least about 0.1 mg/kg, at least about 1 mg/kg, at least about 10 mg/kg, or at least about 20 mg/kg.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Design of IL-10 Muteins

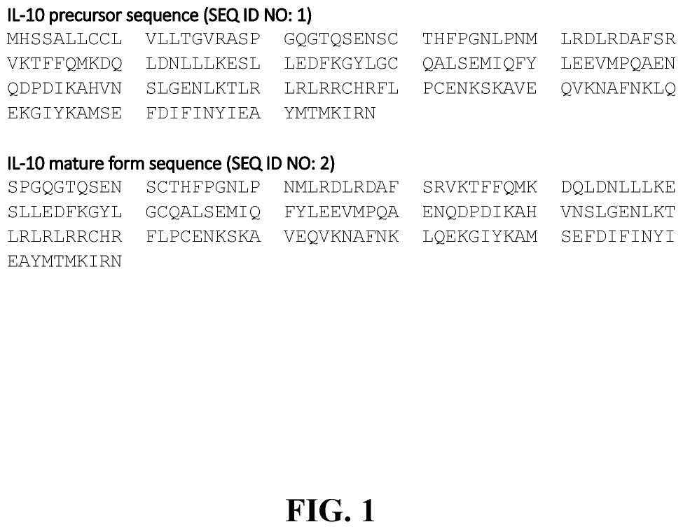

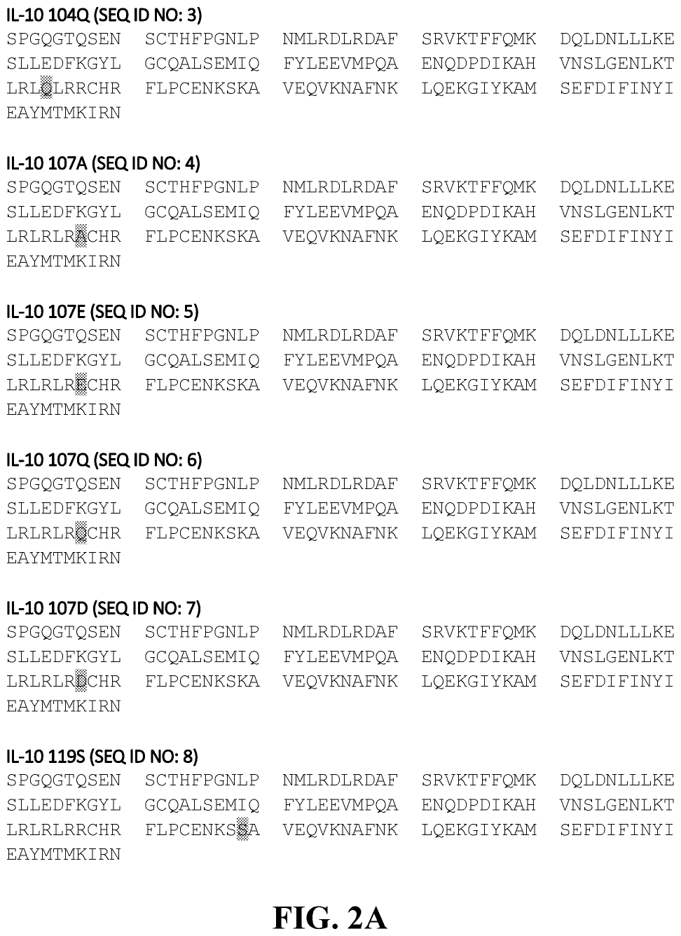

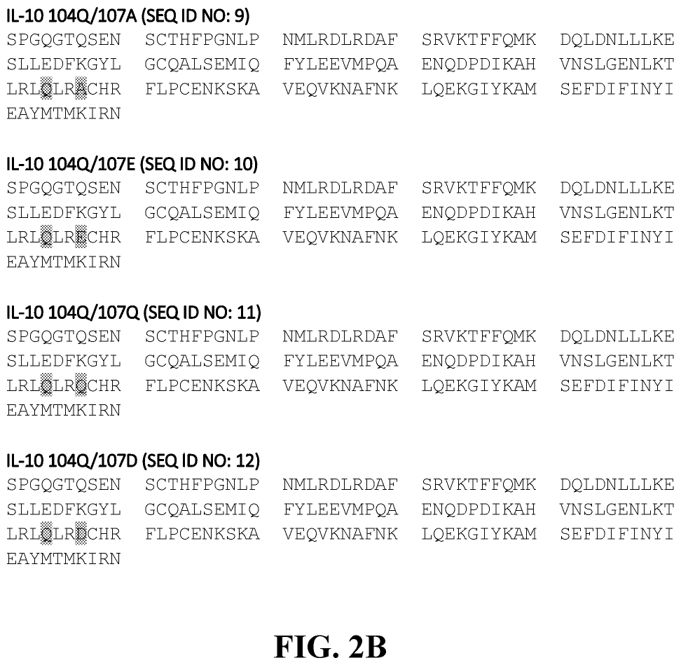

Modulation of IL-10 activity in the presence of heparin has been reported (Blood. 2000; 96 (5): 1879-88). The heparin binding site is a patch of positively charged residues located at the C-terminal end of helix D and the adjacent DE loop involving arginines 102, 104, 106, and 107 and lysines 117 and 119 (J Biol Chem. 2016;291 (6): 3100-13; J Mol Graph Model. 2015; 62:97-104). To investigate the contribution of these residues to heparin binding, substitutions of arginines 104, 107 and lysine 119 were first investigated. Sequences for illustrative IL-10 monomer wild-type (WT) and variants comprising substitutions are depicted in FIGS. 1 and 2A-2B, respectively. It should be noted that the substitution numberings for IL-10 monomer are based on the mature form IL-10 sequence, SEQ ID NO: 2, shown in FIG. 1, and the substitution are label in gray in FIGS. 2A-2B for clarity.

### Example 2: Exemplary IL-10 Muteins in IL10-Fc

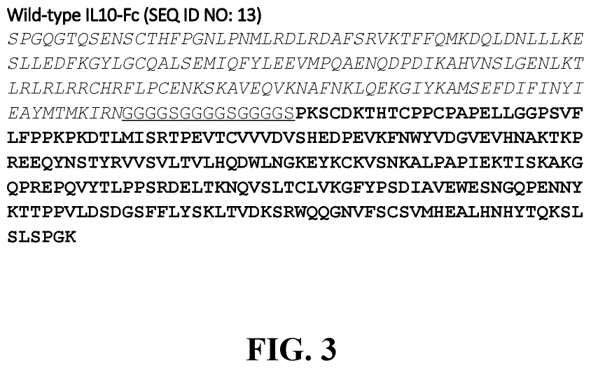

To investigate the effect of substitutions of arginine 104, 107 and lysine 119 on heparin binding, we first generate an IL10 (WT)-Fc (wild-type IL10-Fc) fusion protein having an amino acid sequence SEQ ID NO: 13 shown in FIG. 3, which comprises two identical monomers. Each monomer comprising an IL-10 monomer covalently attached to the N-terminus of a human IgG1 Fc (residue 100 to residue 330 of P01857) via a peptide linker. Synthesis of the nucleic acid molecule encoding IL10 (WT)-Fc fusion protein with an IL-2 signal peptide (MYRMQLLSCIALSLALVTNS, SEQ ID NO: 14) was performed by GeneArt (Thermo Fisher Scientific GENEART) and was constructed into the vector pCDNA3.4 to obtain a recombinant expression vector for expressing the fusion protein in mammalian cells. Afterwards, single point mutations, such as R104Q, R107A, R107E, R107Q and K119S, or multiple point mutations were introduced into the IL10 (WT)-Fc fragment to generate IL10 (R104Q)-Fc, IL10 (R107A)-Fc, IL10 (R107E)-Fc, IL10 (R107Q)-Fc, and IL10 (K119S)-Fc, IL10 (R104Q/R107A)-Fc, IL10 (R104Q/R107Q, and IL10 (R104Q/R107E)-Fc, respectively. Exemplary sequences of the IL-10 variants are in FIGS. 2A-2B.

### Production and Characterization of IL10-Fc Muteins

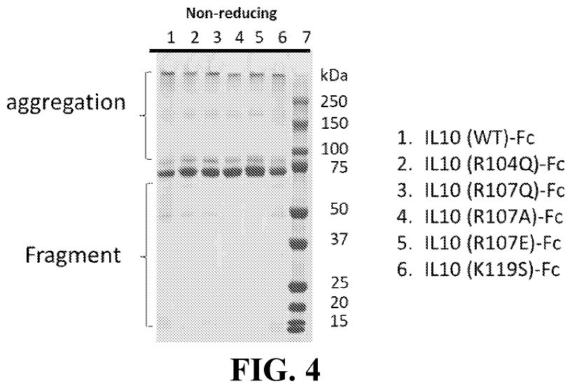

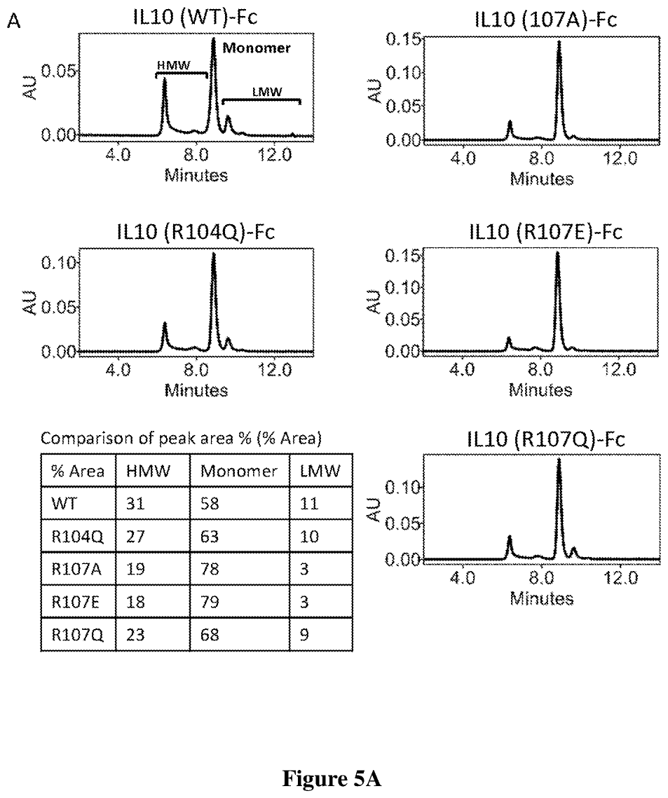

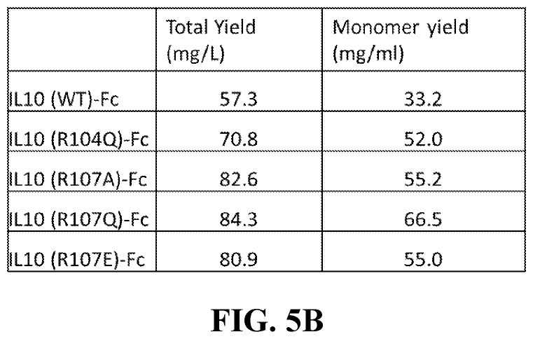

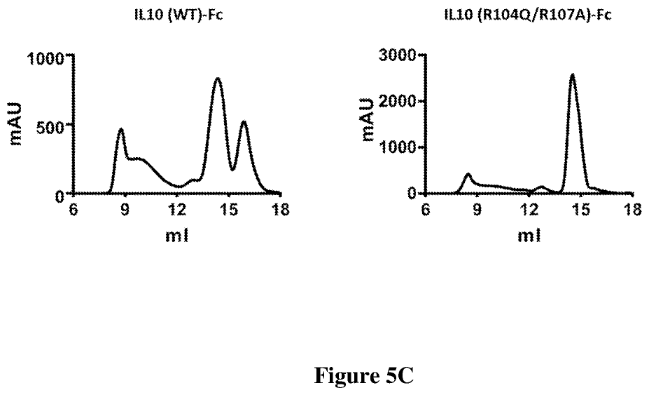

ExpiCHO cells were transfected with the constructed expression vectors according to the manual provided by the manufacturer (ThermoFisher, ExpiCHO™ Expression System Kit A29133). After culturing for 7-8 days, supernatant of the transient expression products was collected, and the IL10 (WT)-Fc and IL10-Fc fusion comprising IL-10 variants were purified using protein A Sepharose and eluted with 0.1 M pH 2.5 Glycine followed by 1M tris pH 9.0 neutralization. The quality of protein A purified proteins were analyzed by non-reducing SDS-PAGE. Surprisingly, as shown in FIG. 4, IL10 (R104Q)-Fc, IL10 (R107A)-Fc, IL10 (R107E)-Fc and IL10 (R107Q)-Fc showed less aggregation when compared to IL10 (WT)-Fc. In contrast, IL10 (K119S)-Fc exhibited similar aggregation when compared to IL10 (WT)-Fc. The IL10-Fc variants purified by protein A were further analyzed for protein aggregation using standard Size Exclusion HPLC (SE-HPLC). 40 μg of antibody was applied to an HPLC (Waters ARC HPLC) and separation was accomplished on a gel filtration column (Waters, XBridge BEH200 SEC 7.8 ×300) using a mobile phase of 0.1 M sodium phosphate, pH 6.8. The antibody peak was monitoring UV absorbance at 280 and the peak area was determined using Empower software. Consistent with SDS-PAGE finding, IL10 (WT)-Fc has significant level of aggregates. IL10 (R104Q)-Fc, IL10 (R107A)-Fc, IL10 (R107E)-Fc, and IL10 (R107Q)-Fc exhibited substantially reduced aggregation and reduced degradation compared to IL10 (WT)-Fc shown in FIG. 5A. This is unexpected, especially for IL10 (R107A)-Fc. Substitution of amino acid arginine in helix at positions 106, 107 and 110 to alanine (R106A, R107A or R110A) are known to destabilize the helix structure of IL-10 (Front Immunol. 2020; 11:1794.). The yield of IL10 (WT)-Fc and muteins in protein A purification is shown in FIG. 5B. The monomer yield of IL10-Fc muteins purified by protein A procedure was at least 1.5-fold higher than that of IL10 (WT)-Fc, apparently due to the increased total protein yield and lower percentage of aggregation. The effects of simultaneous substitutions at R104 and R107 were also investigated. As shown in FIG. 5C, double substitutions of IL10 also showed significant reduced aggregation propensity.

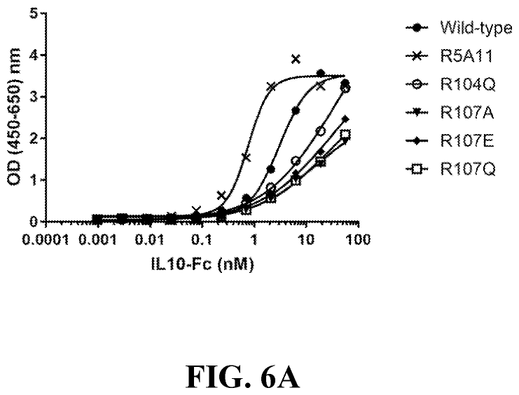

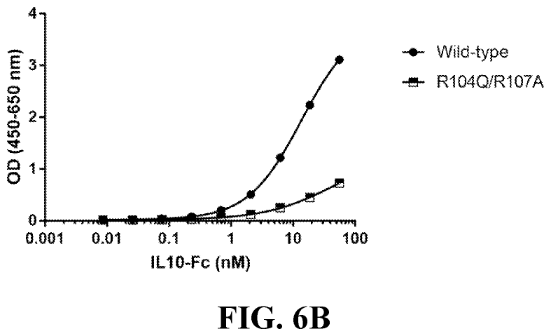

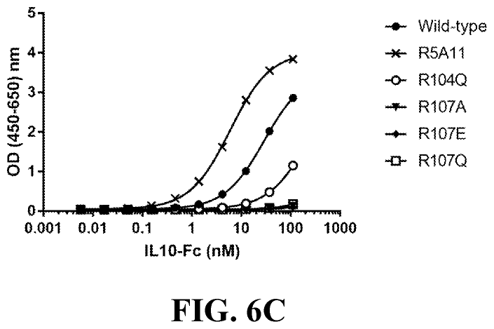

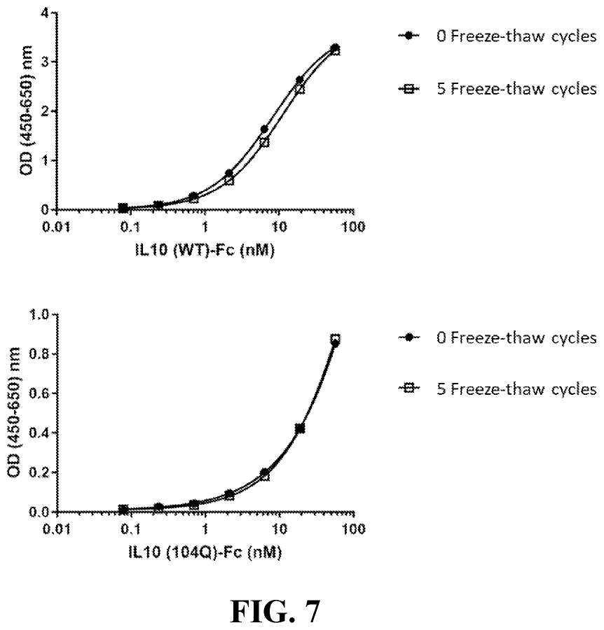

To further characterize IL10-Fc fusion comprising IL10 variants, all fusion proteins were further purified by gel filtration chromatography on a Superdex 200 Increase 10/300 GL column (GE Healthcare) with 1×PBS to remove aggregates. For the binding test, the IL10 (WT)-Fc or muteins were serial diluted with 1×PBS, then 100 μl were added to each well coated with 0.8 μg/ml Recombinant Human IL-10Rα (R&D systems 9100-R1) or 0.1 μg/ml Recombinant Human IL-10Rβ (Sinobiological 10945-H08H). The plate was incubated at 37° C. for 1.5 hours and unbound IL10-Fc were washed away with 1× PBST for three times. Addition of HRP-conjugated anti-human Fc antibody and incubated at 37° C. for one hour. Washed away excess detection antibody and added 3,3′,5,5′-tetramethylbenzidine to each well. After incubation, add equal volume of stopping solution (2M H₂SO₄) and read absorbance at 450/650 nm. As can be seen in FIGS. 6A and 6B, all substitutions of 104 and 107 and simultaneous substitution at R104 and R107 exhibited significantly reduced human IL-10Rα binding when compared to IL10 (WT)-Fc. In contrast, R5A11, an IL-10 mutein with multiple substitutions described by Gorby et al. at N15, N92, K99, and F111 (Sci Signal. 2020 Sep. 15;13 (649): eabc0653) showed enhanced Human IL-10Rα and IL-10Rβ binding. The binding of IL10-Fc fusion comprising IL10 variants to IL-10Rβ were also significantly reduced in ELISA assay (FIG. 6C). We also used the IL-10Rα binding ELISA to investigate the of freeze-thaw stability of IL10 (R104Q)-Fc. IL10-Fc fusion protein in PBS was freeze-thawed five times. The cycles consisted of placing samples at −70° C. more than 24 hours. The next day, the sample were thawed at room temperature for at least 1 hour. As can be seen in FIG. 7, five freeze-thawing treatment had a negative influence on the binding affinity of IL10 (WT)-Fc to IL-10Rα. In contrast, the binding of IL10 (R104Q)-Fc to human IL-10Rα was not changed after five freeze-thawing treatment.

### STAT3 Activation by IL10-Fc Muteins

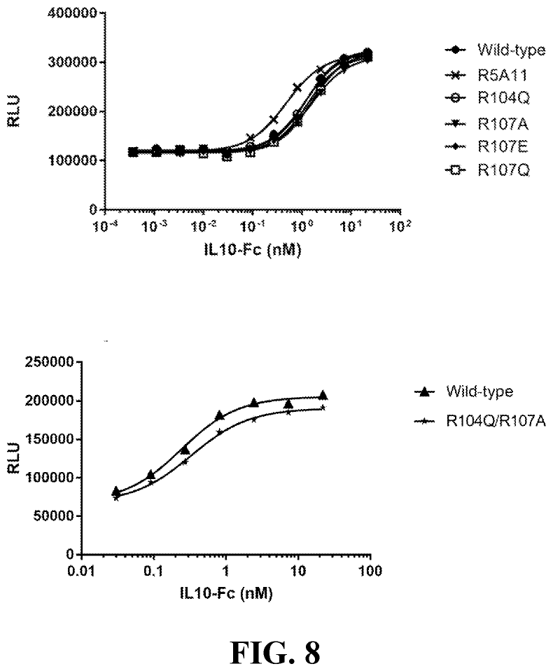

A cell-based bioassay was established to study STAT3 activation by IL-10. Hela cells expresses endogenous IL-10Rβ but not IL-10Rα. Therefore, HeLa IL10Rα-STAT3 luciferase reporter cells which stably expresses human IL-10Rα and a STAT3 Firefly luciferase reporter gene under the transcriptional control of the STAT3 were generated using standard lentivirus technique. STAT3 Firefly luciferase reporter lentivirus was purchased from Cellomics Technology (PLV-10065-50). Lentivirus encoding IL-10Rα (NM_001558) was generated using transfer vector pLAS5w.Phyg and standard method. Puromycin and hygromycin were used to select HeLa IL10Rα-STAT3 stable cells. For the STAT3 activation assay, IL10Rα-STAT3 stable cell were seeded into a white solid-bottom 96-well microplate in 100 μl of growth medium at 5×10⁴ cells/well. Cells were then incubated at 37° C. in a CO₂ incubator for another 6 hour and then stimulated with different concentrations of IL10 (WT)-Fc or IL10-Fc fusion comprising IL10 variants. After another 18 hr culture, luciferase activity was determined by ONE-Glo™ Luciferase Assay System (Promega E6120) according to the manufacturer's instructions. As shown in FIG. 8, the activity of IL10-Fc fusion comprising IL10 variants were comparable to IL10 (WT)-Fc. IL10 (R5A11)-Fc showed better activity which is consistent with literature's observation.

### CD8 T Cell Activation by IL10-Fc Muteins

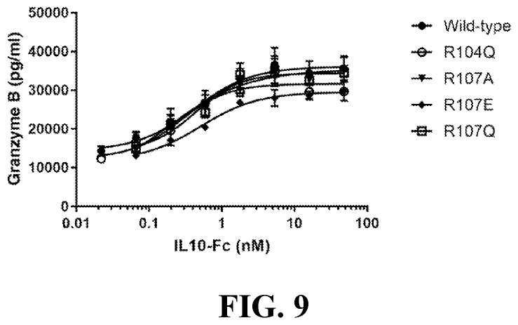

Stimulation of CD8+ T cells is one of the anti-tumor features of IL-10. IL-10R on CD8+ T cells was necessary and sufficient for increased tumor-resident CD8+ T cell proliferation, and increased activity in IL-10-treated tumor-bearing mice (Cancer Res. 2012; 72 (14): 3570-81.). Thus, the functional activity of IL10-Fc fusion comprising IL10 variants on CD8+ T cells was assessed using primary CD8+ T cells. CD8+ T cells were isolated from PBMC using CD8 microbeads (Miltenyi Biotec: 130-045-201) and stimulated for 72 hours with T cell TransAct (Miltenyi Biotec: 130-111-160) in AIM-V medium (Thermo Fisher Scientific: 12055083). After stimulation, activated CD8+ T cells were incubated with IL10-Fc fusion proteins. Secreted granzyme B was determined by ELISA (R&D systems DY2906-05) according to the manufacturer's instructions. IL10 (R104Q)-Fc and IL10 (R107Q)-Fc showed comparable stimulation activity compared to IL10 (WT)-Fc (FIG. 9) while the human IL-10Rα binding activity of IL10 (R104Q)-Fc, IL10 (R107A)-Fc, IL10 (R107E)-Fc and IL10 (R107Q)-Fc is weaker than IL10 (WT)-Fc. These data suggest that IL10 (R104Q)-Fc, IL10 (R107A)-Fc, IL10 (R107E)-Fc and IL10 (R107Q)-Fc can function like IL10 (WT)-Fc in human CD8+ T cell assays despite receptor binding being greatly diminished.

### IL10-Fc-VEGF Trap

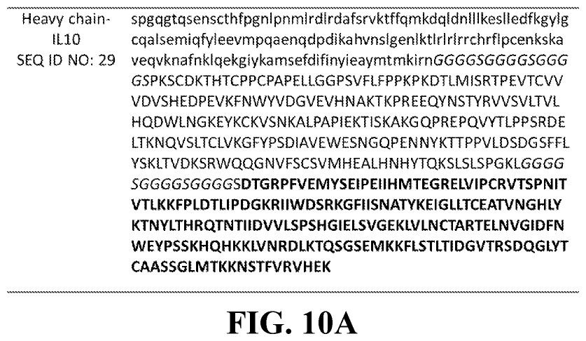

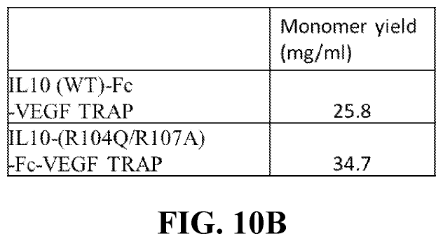

Reduced aggregation potency of IL10 muteins were also observed in another fusion example. A vascular endothelial growth factor (VEGF) trap was fused to the C terminus of IL10 wild-type or muteins. The sequence of VEGF trap consists of the Ig domain 2 from VEGFR1, which is fused to Ig domain from VEGFR2 (SEQ ID NO: 29) (FIG. 10A). Fusion proteins were expressed, purified, and analyzed by SE HPLC as previously described. As can be seen in FIG. 10B, IL-10 (R104Q/R107A)-Fc-VEGF trap showed reduced aggregation when compared to IL-10 (WT)-Fc-VEGF trap fusion protein.

### Example 3: Antibody-IL10 Format

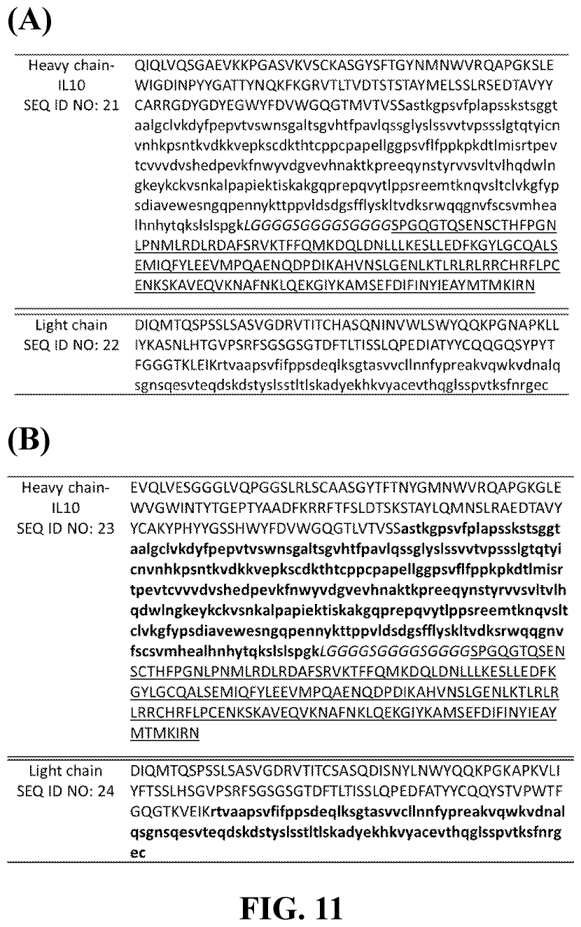

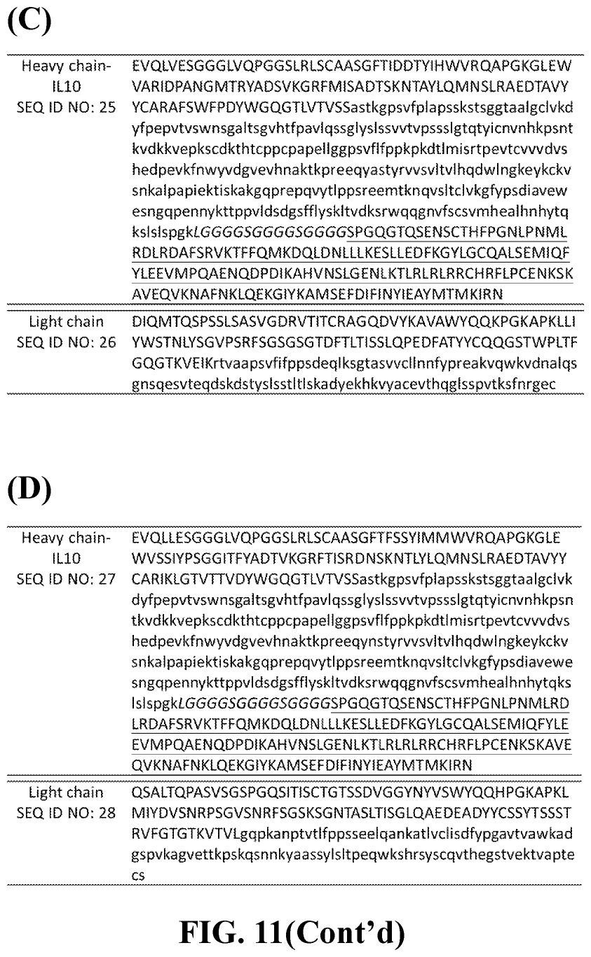

To investigate if the improved manufacturability of IL-10 muteins could be observed when N-terminus of IL-10 muteins are fused to the C-terminus of another protein, Antibody-IL10 fusion constructs based on monoclonal antibodies including 6D4H22-IL10 (SEQ ID NO: 21-22), were generated by standard cloning technique. IL10 was fused to the C-terminus of the respective antibodies via a peptide linker as shown in FIG. 11 to generate an antibody-IL10 fusion proteins. Details about the generation, affinity maturation and characterization of 6D4H22 (anti-CSFIR) antibody can be found in the examples appended to PCT publication no. WO2020242950A1, which is incorporated herein by reference. Next, the Antibody-IL10 fusion protein containing single substitutions (R104Q, R107A, R107E, or R107Q) or the double substitutions (R104Q/R107A, R104Q/R107D, R104Q/R107E or R104Q/R107Q) in the IL-10 sequence were generated by standard cloning techniques. Sequences for the exemplary IL-10 muteins are depicted in FIG. 2.

### Aggregation of 6D4H22-IL10 Muteins

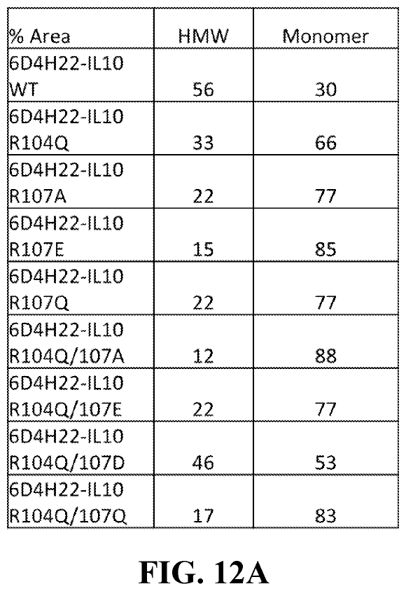

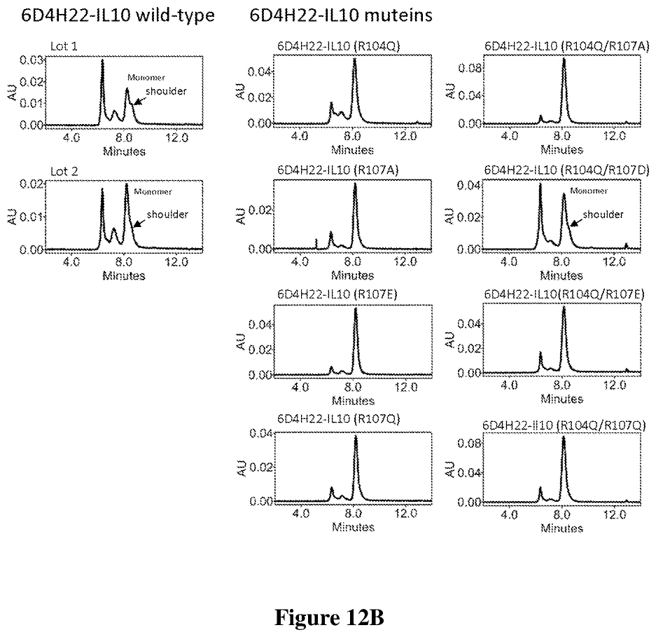

The aggregate content of Protein A-eluted 6D4H22-IL10 (WT) and various 6D4H22-IL10 mutein fusion proteins was analyzed using SE HPLC as described above. All 6D4H22-IL10 muteins exhibited production advantages over the 6D4H22-IL10 (WT). Firstly, the percentage of monomer is greatly increased in various 6D4H22-IL10 mutein fusion proteins (FIG. 12A). Secondly, no heterogeneity of the main peak observed in 6D4H22-IL10 single muteins including R104Q, R107A, R107E, R107Q and double muteins R104Q/R107A, R104Q/R107Q, R104Q/R107E (FIG. 12B). The shoulder of monomer was still observed in the double muteins R104Q/R107D.

### IL-10Rα Binding

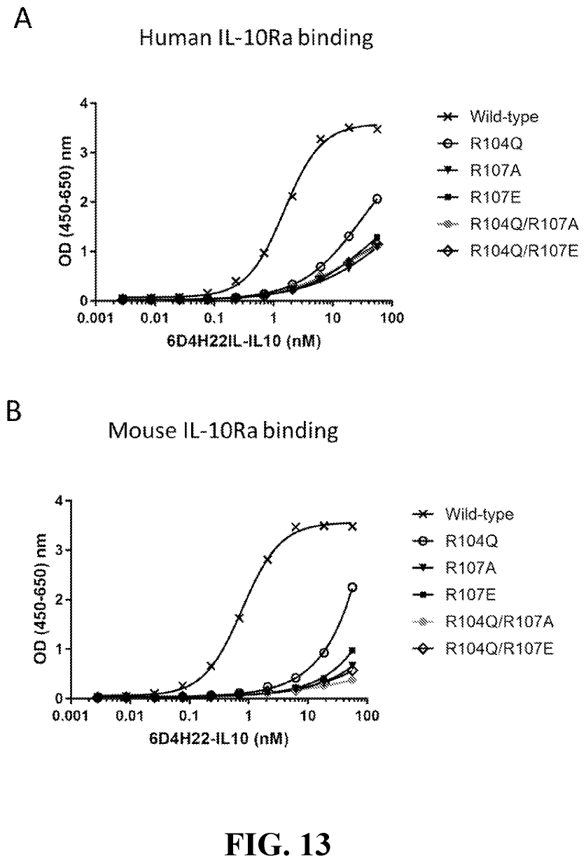

We also investigated whether these IL10 muteins exhibit lower binding to Human IL-10Rα when fused to C-terminus of another protein. All Protein A-eluted 6D4H22-IL10 (WT) and various 6D4H22-IL10 mutein fusion proteins were further purified by gel filtration chromatography on a Superdex 200 Increase 10/300 GL column (GE Healthcare) with 1× PBS to remove aggregates. For the binding test, the 6D4H22-IL10 (WT) and muteins were serial diluted with 1×PBS, then 100 μl were added to the wells coated with recombinant human IL-10Rα (R&D systems 274-R1). As can be seen in FIG. 13A, all substitutions of 104 and 107 in IL-10 moiety of 6D4H22-IL10 exhibited significantly reduced human IL-10Rα binding when compared to 6D4H22-IL-10 (WT) (FIG. 11A). The binding activity of 6D4H22-IL10 mutein fusion proteins to mouse IL-10Rα (R&D systems 7704-MR-025/CF) was also reduced with a similar pattern to the binding of same muteins to human IL-10Rα (FIG. 13B). The data suggested that the reduced receptor binding is a characteristic of these IL-10 muteins and would not be changed when IL-10 is fused to C-terminus or N-terminus of another polypeptide.

### STAT3 Activation by 6D4H22-IL10 Muteins

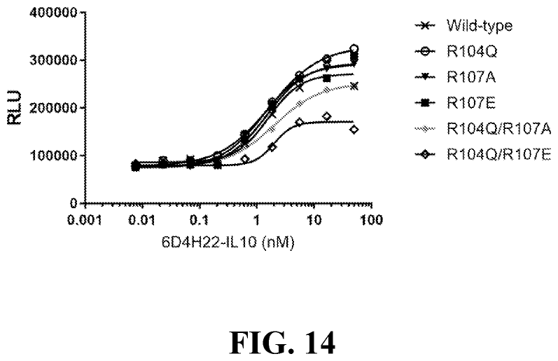

Briefly, HeLa IL10Rα-STAT3 luciferase reporter cells were seeded into a white solid-bottom 96-well microplate in 100 μl of growth medium at 5×10⁴ cells/well. Incubate cells at 37° C. in a CO₂ incubator for another 6 hours and then stimulate cells with different concentrations of 6D4H22-IL10 (WT) and 6D4H22-IL10 mutein fusion proteins. After another 18 hr culture, luciferase activity was determined by ONE-Glo™ Luciferase Assay System (Promega E6210). As shown in FIG. 14, 6D4H22-IL10 mutein fusion proteins retained biological activity while the binding of fusion protein to IL-10Rα was greatly reduced.

### CD8+ T Cell Activation by 6D4H22-IL10 Muteins

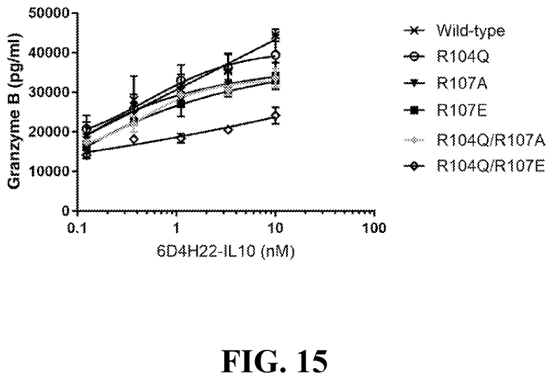

The functional activity of 6D4H22-IL10 variants on CD8+ T cells was assessed using primary CD8+ T cells. CD8+ T cells were isolated from PBMC using CD8 microbeads (Miltenyi Biotec: 130-045-201) and stimulated for 72 hours with T cell TransAct (Miltenyi Biotec: 130-111-160) in AIM-V medium (Thermo Fisher Scientific: 12055083). After stimulation, activation CD8+ T cells were incubated with 6D4-IL10 variants. Secreted granzyme B were determined by ELISA (R&D systems DY2906-05) Consistent with IL-10 muteins in IL10-Fc format, 6D4H22-IL10 (R104Q) showed comparable stimulation activity compared to 6D4H22-IL10 (WT) (FIG. 15).

Other Fusions with IL-10 at C-Terminal

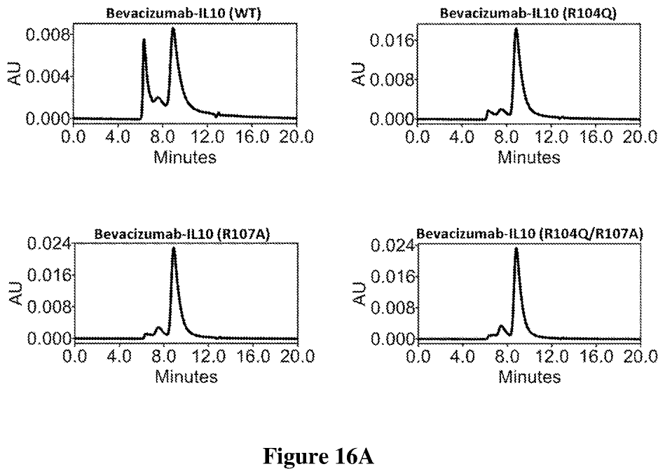

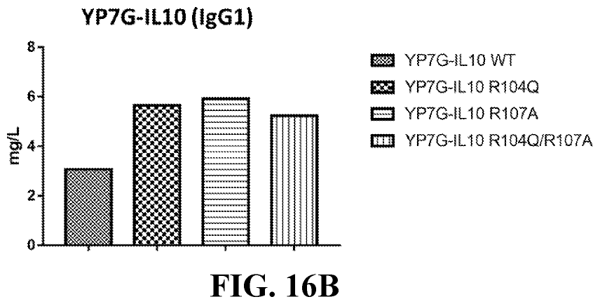

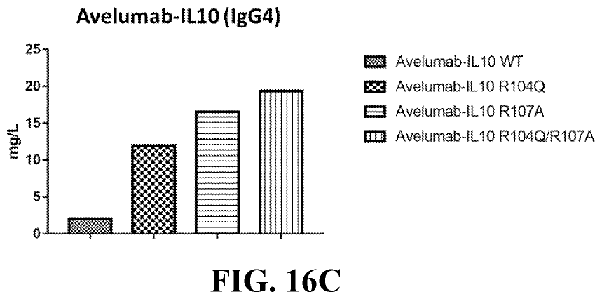

IL10 (WT) and muteins were fused to the C-terminus of Bevacizumab (IgG1, SEQ ID NO: 23-24), YP7G (IgG4, SEQ ID NO: 25-26) or Avelumab (IgG1, SEQ ID NO: 27-28) via a peptide linker (FIG. 11(B)-(D)) to generate antibody-IL10 fusion proteins. Details about the generation, affinity maturation and characterization of YP7G (anti-PD-L1) antibody can be found in the examples appended to PCT publication no. WO2021231741A2, which is incorporated herein by reference. The amino acid sequence of Avelumab and Bevacizumab can be found in WO2013079174 (A09-246-2) and WO2013181586, respectively. Bevacizumab and Avelumab were obtained from construction of Antibody-IL-10 fusions. All fusion proteins were expressed in CHO—S and purified by protein A beads as previous described. In FIG. 16A, size-exclusion chromatography results showed that the level of aggregation was greatly reduced when Bevacizumab was fused to IL10 muteins R104Q, R107A or R104/Q/R107A. Also, the monomer yield of anti-PD-L1 antibodies, YP7G and Avelumab, were also significantly increased in fusions with IL10 muteins when compared to antibody-IL10 (WT) fusion (FIGS. 16B and 16C).

### Example 4: Pharmacokinetics of IL-10 Fusion Proteins

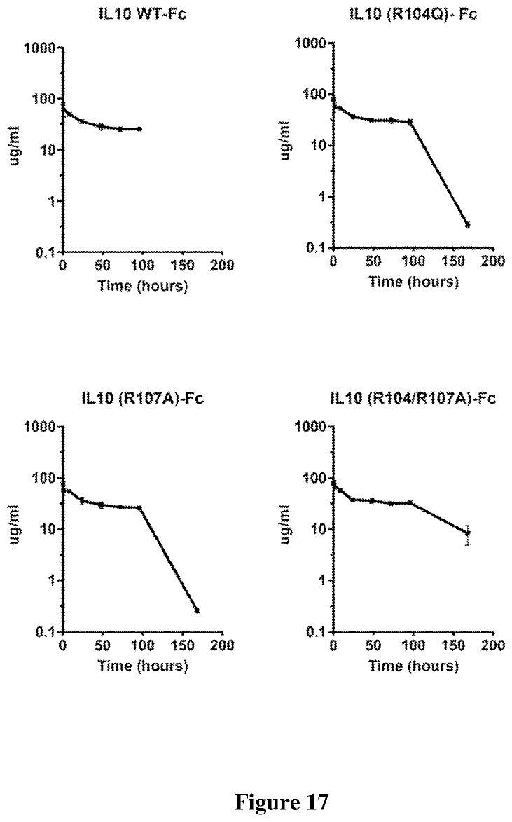

The pharmacokinetic profiles of IL-10 (R104Q)-Fc, IL-10 (R107A)-Fc and IL-10 (R104Q/R107A)-Fc fusion proteins were compared to the profile of IL-10 (WT)-Fc. Pharmacokinetic study was conducted in nonfasted female C57BL6 mice after i.v. administration (10 mg/kg). Mice received either IL-10 (WT)-Fc or IL-10 mutein-Fc fusion proteins that was formulated in PBS (three mice per group). Serial blood samples (10 μl) were harvested by microsampling (Pharm Res. 2014;31 (7): 1823-33.) on the tail vein at predose, 0.25, 1, 8, 24, 48, 72 and 96 hours post dose diluted with 90 μl PBS with 1% BSA. At the end of the studies (196 hours), serum samples were also collected via cardiac puncture. Samples were stored at −80° C. until analysis for hIL-10 Fc concentrations. Intact protein concentrations were determined in an ELISA assay in which the IL-10 Fc fusions is captured by an anti-IL10 antibody (Biolegend 506802) and detected by anti-human Fc (Abcam Ab97225). Results of a representative experiment are shown in FIG. 17. A time course of the concentration of the IL-10 fusion over 196 hours showed substantially extended half-life of IL-10 mutein-Fc fusion proteins (R104Q, R107A or R104Q/R107A) when compared to IL-10 (WT)-Fc. At 196 hours, the concentration of IL-10 (WT)-Fc was below detection limit, while IL-10 (R104Q)-Fc, IL-10 (R107A)-Fc and IL-10 (R104Q/R107A)-Fc were still detectable.

Notwithstanding the appended claims, the disclosure set forth herein is also defined by the following clauses, which may be beneficial alone or in combination, with one or more other causes or embodiments. Without limiting the foregoing description, certain non-limiting clauses of the disclosure numbered as below are provided, wherein each of the individually numbered clauses may be used or combined with any of the preceding or following clauses. Thus, this is intended to provide support for all such combinations and is not necessarily limited to specific combinations explicitly provided below:

While the foregoing disclosure of the present invention has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the spirit and purview of this disclosure and the appended claims. Further, one of skill in the art will recognize a number of equivalent methods and procedure to those described herein. All such equivalents are to be understood to be within the scope of the present disclosure and are covered by the appended claims.

The disclosures of all publications, patent applications, patents, or other documents mentioned herein are expressly incorporated by reference in their entirety for all purposes to the same extent as if each such individual publication, patent, patent application or other document were individually specifically indicated to be incorporated by reference herein in its entirety for all purposes and were set forth in its entirety herein. In case of conflict, the present specification, including specified terms, will control.

### REFERENCE

1. Saraiva M, Vieira P, O'Garra A. Biology and therapeutic potential of interleukin-10. J Exp Med. 2020 Jan. 6;217 (1): e20190418.
2. Ni G, Zhang L, Yang X, et al. Targeting interleukin-10 signalling for cancer immunotherapy, a promising and complicated task. Hum Vaccin Immunother. 2020; 16 (10): 2328-2332.
3. Oft M. Immune regulation and cytotoxic T cell activation of IL-10 agonists-Preclinical and clinical experience. Semin Immunol. 2019; 44:101325.
4. Rallis K S, Corrigan A E, Dadah H, et al. Cytokine-based Cancer Immunotherapy: Challenges and Opportunities for IL-10. Anticancer Res. 2021;41 (7): 3247-3252.
5. Mumm J B, Emmerich J, Zhang X, Chan I, Wu L, Mauze S, Blaisdell S, Basham B, Dai J, Grein J, Sheppard C, Hong K, Cutler C, Turner S, LaFace D, Kleinschek M, Judo M, Ayanoglu G, Langowski J, Gu D, Paporello B, Murphy E, Sriram V, Naravula S, Desai B, Medicherla S, Seghezzi W, McClanahan T, Cannon-Carlson S, Beebe A M, Oft M. IL-10 elicits IFNγ-dependent tumor immune surveillance. Cancer Cell. 2011 Dec. 13;20 (6): 781-96.
6. Naing A, Infante J R, Papadopoulos K P, et al. PEGylated IL-10 (Pegilodecakin) Induces Systemic Immune Activation, CD8+ T Cell Invigoration and Polyclonal T Cell Expansion in Cancer Patients. Cancer Cell. 2018;34 (5): 775-791.e3.
7. Guo Y, Xie Y Q, Gao M, et al. Metabolic reprogramming of terminally exhausted CD8+ T cells by IL-10 enhances anti-tumor immunity. Nat Immunol. 2021;22 (6): 746-756.
8. Trachsel, E., Bootz, F., Silacci, M. et al. Antibody-mediated delivery of IL-10 inhibits the progression of established collagen-induced arthritis. Arthritis Res Ther 9, R9 (2007).
9. Schwager K, Kaspar M, Bootz F, Marcolongo R, Paresce E, Neri D, Trachsel E. Preclinical characterization of DEKAVIL (F8-IL10), a novel clinical-stage immunocytokine which inhibits the progression of collagen-induced arthritis. Arthritis Res Ther. 2009; 11 (5): R142.
10. Westerhof L B, Wilbers R H, Roosien J, van de Velde J, Goverse A, Bakker J, Schots A. 3D domain swapping causes extensive multimerisation of human interleukin-10 when expressed in planta. PLOS One. 2012; 7 (10): e46460.
11. Ding Y, Qin L, Kotenko S V, Pestka S, Bromberg J S. A single amino acid determines the immunostimulatory activity of interleukin 10. J Exp Med. 2000 Jan. 17;191 (2): 213-24.
12. U.S. Pat. No. 7,749,490B2
13. Saxton R A, Tsutsumi N, Su L L, Abhiraman G C, Mohan K, Henneberg L T, Aduri N G, Gati C, Garcia K C. Structure-based decoupling of the pro- and anti-inflammatory functions of interleukin-10. Science. 2021 Mar. 19;371 (6535): eabc8433.
14. Gorby C, Sotolongo Bellón J, Wilmes S, Warda W, Pohler E, Fyfe P K, Cozzani A, Ferrand C, Walter M R, Mitra S, Piehler J, Moraga I. Engineered IL-10 variants elicit potent immunomodulatory effects at low ligand doses. Sci Signal. 2020 Sep. 15;13 (649): eabc0653.

1. An IL-10 mutein comprising a substitution on amino acids in position 104, position 107, and a combination thereof, relative to amino acids of a wild-type IL-10.

2. The IL-10 mutein of claim 1, wherein the wild-type IL-10 comprises an amino acid sequence having at least 80%, preferably at least 90%, and more preferably at least 95%, identity with SEQ ID NO: 2.

3. The IL-10 mutein of claim 1, wherein the substitution comprises:
a. R104Q;
b. any one of R107A, R107E, R107Q and R107D; or
c. a combination thereof.

4. The IL-10 mutein of claim 3, wherein the substitution comprises R104Q/R107A, R104Q/R107E, R104Q/R107Q or R104Q/R107D.

5. The IL-10 mutein of claim 1, wherein the IL-10 mutein is a monomer or dimer.

6. The IL-10 mutein of claim 1, further comprising a signal peptide.

7. The IL-10 mutein of claim 6, wherein the signal peptide comprises the amino acid sequence of SEQ ID NO: 14.

8. A fusion protein, comprising
a polypeptide which binds to a target protein, wherein the polypeptide comprises an antibody or
a fragment thereof, an antagonist, a receptor or a ligand of the target protein, or a protein-Trap; and
the IL-10 mutein according to of claim 1, fused to the polypeptide.

9. The fusion protein of claim 8, wherein the polypeptide is fused to the IL-10 mutein via a linker.

10. The fusion protein of claim 9, wherein the linker comprises the amino acid sequence of SEQ ID NO: 15-20.

11. The fusion protein of claim 8, wherein the IL-10 mutein is fused to a N-terminal or a C-terminal of the polypeptide.

12. (canceled)

13. The fusion protein of claim 8, wherein the amino acid sequence has at least 80% identity to a sequence selected from:
a. SEQ ID NO: 13;
b. SEQ ID NO: 21 and SEQ ID NO: 22;
c. SEQ ID NO: 23 and SEQ ID NO: 24;
d. SEQ ID NO: 25 and SEQ ID NO: 26;
e. SEQ ID NO: 27 and SEQ ID NO: 28; or
f. SEQ ID NO: 29.

14. The fusion protein of claim 8, wherein the protein-Trap comprises a vascular endothelial growth factor-Trap (VEGF-Trap) including aflibercept.

15. The fusion protein of claim 9, wherein the amino acid sequence has at least 80% identity to SEQ ID NO: 29.

16. The fusion protein of claim 8, wherein: (i) the antibody is a human, humanized, or chimeric antibody; (ii) the antibody is a full length antibody of class IgG, optionally, wherein the class IgG antibody has an isotype selected from IgG1, IgG2, IgG3, and IgG4; (iii) the antibody comprises an Fc region variant, optionally an Fc region variant that alters effector function and/or a variant that alters antibody half-life; (iv) the antibody is an antibody fragment, optionally selected from the group consisting of F(ab′)₂, Fab′, Fab, Fv, single domain antibody (VHH), and scFv; (v) the antibody comprises an immunoconjugate, optionally, wherein the immunoconjugate comprises a therapeutic agent for treatment of a CSFIR-mediated, PDL1-mediated, PD1-mediated or VEGF-mediated disease or condition; or (vi) the antibody is a multi-specific antibody, optionally a bispecific antibody.

17. The fusion protein of claim 8, wherein the polypeptide is a half-life extending moiety.

18. The fusion protein of claim 16, wherein the half-life extending moiety comprises a IgG constant domain or fragment thereof, a human serum albumin (HSA), or a albumin-binding polypeptides or a residue.

19. An isolated host cell comprising:
an isolated polynucleotide or vector encoding the IL-10 mutein of claim 1.

20. The isolated host cell of claim 19; wherein the host cell is selected from a group consisting of Chinese hamster ovary (CHO) cell, a myeloma cell comprising Y0, NS0 or Sp2/0, a monkey kidney cell comprising COS-7, a human embryonic kidney line comprising 293, a baby hamster kidney cell (BHK), a mouse Sertoli cell comprising TM4, an African green monkey kidney cell comprising VERO-76, a human cervical carcinoma cell (HELA), a canine kidney cell, a human lung cell comprising W138, a human liver cell comprising Hep G2, a mouse mammary tumor cell, a TR1 cell, a Medical Research Council 5 (MRC 5) cell, and a FS4 cell.

21. A method of producing an IL-10 mutein or a fusion protein, the method comprising the step of culturing the isolated host cell of claim 19, wherein the IL-10 mutein or a fusion protein is produced.

22. A pharmaceutical composition comprising an IL-10 mutein of claim 1 wherein the pharmaceutically acceptable carrier is al diluent or a excipient.

23. (canceled)

24. The method of claim 21, further comprising the step of administering to a subject a therapeutically effective amount of the composition to a subject in need thereof.


SEQUENCE LISTING (29 sequences)

SEQ ID NO: 1 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - protein, 160 aa, Homo sapiens
  SIGNAL 1..18
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 3 - protein, 160 aa, synthetic construct
  REGION 1..160: note = R104Q
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLQLRRCHRFLPCENKSKAVEQVKNAFNKLQEKGIYKAMSEFDIFINYIEAYMTMKIRN

SEQ ID NO: 4 - protein, 160 aa, synthetic construct
  REGION 1..160: note = R107A
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRACHRFLPCENKSKAVEQVKNAFNKLQEKGIYKAMSEFDIFINYIEAYMTMKIRN

SEQ ID NO: 5 - protein, 160 aa, synthetic construct
  REGION 1..160: note = R107E
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRECHRFLPCENKSKAVEQVKNAFNKLQEKGIYKAMSEFDIFINYIEAYMTMKIRN

SEQ ID NO: 6 - protein, 160 aa, synthetic construct
  REGION 1..160: note = R107Q
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRQCHRFLPCENKSKAVEQVKNAFNKLQEKGIYKAMSEFDIFINYIEAYMTMKIRN

SEQ ID NO: 7 - protein, 160 aa, synthetic construct
  REGION 1..160: note = R107D
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRDCHRFLPCENKSKAVEQVKNAFNKLQEKGIYKAMSEFDIFINYIEAYMTMKIRN

SEQ ID NO: 8 - protein, 160 aa, synthetic construct
  REGION 1..160: note = K119S
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSSAVEQVKNAFNKLQEKGIYKAMSEFDIFINYIEAYMTMKIRN

SEQ ID NO: 9 - protein, 160 aa, synthetic construct
  REGION 1..160: note = R104Q/R107A
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLQLRACHRFLPCENKSKAVEQVKNAFNKLQEKGIYKAMSEFDIFINYIEAYMTMKIRN

SEQ ID NO: 10 - protein, 160 aa, synthetic construct
  REGION 1..160: note = R104Q/R107E
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLQLRECHRFLPCENKSKAVEQVKNAFNKLQEKGIYKAMSEFDIFINYIEAYMTMKIRN

SEQ ID NO: 11 - protein, 160 aa, synthetic construct
  REGION 1..160: note = R104Q/R107Q
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLQLRQCHRFLPCENKSKAVEQVKNAFNKLQEKGIYKAMSEFDIFINYIEAYMTMKIRN

SEQ ID NO: 12 - protein, 160 aa, synthetic construct
  REGION 1..160: note = R104Q/R107D
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLQLRDCHRFLPCENKSKAVEQVKNAFNKLQEKGIYKAMSEFDIFINYIEAYMTMKIRN

SEQ ID NO: 13 - protein, 406 aa, synthetic construct
  REGION 1..406: note = WT IL10-Fc
  DOMAIN 1..160: note = Wild-type IL10-Fc
  DOMAIN 161..175: note = linker
  DOMAIN 176..406: note = Fc
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSGGGGSGGGGSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 20 aa, synthetic construct
  REGION 1..20: note = signal
MYRMQLLSCIALSLALVTNS

SEQ ID NO: 15 - protein, 5 aa, synthetic construct
  REGION 1..5: note = L1
  REPEAT 1..5: note = 1-6 repeats
GGGGS

SEQ ID NO: 16 - protein, 5 aa, synthetic construct
  REGION 1..5: note = L2
  REPEAT 1..5: note = 1-6 repeats
SSSSG

SEQ ID NO: 17 - skipped

SEQ ID NO: 18 - protein, 5 aa, synthetic construct
  REGION 1..5: note = L4
  REPEAT 1..5: note = 1-6 repeats
EAAAK

SEQ ID NO: 19 - skipped

SEQ ID NO: 20 - protein, 7 aa, synthetic construct
  REGION 1..7: note = L6
  REPEAT 1..7: note = 1-6 repeats
  SITE 7: note = MISC_FEATURE - X is G or S
ENLYFQX

SEQ ID NO: 21 - protein, 628 aa, synthetic construct
  REGION 1..628: note = Heavy chain-IL10 of 6D4H22-IL10
QIQLVQSGAEVKKPGASVKVSCKASGYSFTGYNMNWVRQAPGKSLEWIGDINPYYGATTYNQKFKGRVTLTVDTSTSTAYMELSSLRSEDTAVYYCARRGDYGDYEGWYFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLGGGGSGGGGSGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 22 - protein, 214 aa, synthetic construct
  REGION 1..214: note = Light chain of 6D4H22
DIQMTQSPSSLSASVGDRVTITCHASQNINVWLSWYQQKPGNAPKLLIYKASNLHTGVPSRFSGSGSGTDFTLTISSLQPEDIATYYCQQGQSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 628 aa, synthetic construct
  REGION 1..628: note = Heavy chain-IL10 of Bevacizumab-IL10
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLGGGGSGGGGSGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 24 - protein, 214 aa, synthetic construct
  REGION 1..214: note = Light chain of Bevacizumab
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 622 aa, synthetic construct
  REGION 1..622: note = Heavy chain-IL10 of YP7G-IL10
EVQLVESGGGLVQPGGSLRLSCAASGFTIDDTYIHWVRQAPGKGLEWVARIDPANGMTRYADSVKGRFMISADTSKNTAYLQMNSLRAEDTAVYYCARAFSWFPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLGGGGSGGGGSGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 26 - protein, 214 aa, synthetic construct
  REGION 1..214: note = Light chain of YP7G
DIQMTQSPSSLSASVGDRVTITCRAGQDVYKAVAWYQQKPGKAPKLLIYWSTNLYSGVPSRFSGSGSGTDFTLTISSLQPEDFATYYCQQGSTWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 625 aa, synthetic construct
  REGION 1..625: note = Heavy chain-IL10 of Avelumab-IL10
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLGGGGSGGGGSGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 28 - protein, 216 aa, synthetic construct
  source 1..216: note = Light chain of Avelumab
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 29 - protein, 626 aa, synthetic construct
  source 1..626: note = IL10-Fc-VEGF TRAP
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSGGGGSGGGGSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLGGGGSGGGGSGGGGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK